(19) United States
(12) Patent Application Publication
(10) Pub. No.: US 2022/0033479 A1
(43) Pub. Date: Feb. 3, 2022

(54) ANTI-HEPATITIS C VIRUS ANTIBODIES

(71) Applicants: Meital GAL-TANAMY, Kfar Vradim (IL); Gur YAARI, Givat Shmuel (IL); Shiri ELMEDVI, Kibbutz Shamir (IL); Sivan ELIYAHU, Kibbutz Yehiam (IL); Oz SHARABI, Holon (IL); BAR ILAN UNIVERSITY, Ramat Gan (IL)

(72) Inventors: Meital GAL-TANAMY, Kfar Vradim (IL); Gur YAARI, Givat Shmuel (IL); Shiri ELMEDVI, Kibbutz Shamir (IL); Sivan ELIYAHU, Kibbutz Yehiam (IL); Oz SHARABI, Holon (IL)

(73) Assignee: BAR ILAN UNIVERSITY, Ramat Gan (IL)

(21) Appl. No.: 17/309,796

(22) PCT Filed: Dec. 19, 2019
(86) PCT No.: PCT/IL2019/051392

§ 371 Date: Jun. 18, 2021

Related U.S. Application Data
(60) Provisional application No. 62/781,634, filed on Dec. 19, 2018.

(51) Int. Cl. C07K 16/10; A61K 39/42; G01N 33/576; A61P 31/14

(52) U.S. Cl. CPC C07K 16/109; A61K 39/42; G01N 33/5767; C07K 2317/76; G01N 2469/10; C07K 2317/622; C07K 2317/21; A61P 31/14

(57) ABSTRACT

Provided are isolated monoclonal antibodies or any antigen-binding fragment thereof which bind to hepatitis C virus E2 protein (HCV E2). In particular the presently claimed subject matter concerns neutralizing anti HCV E2 antibodies, and their use for treating HCV infection. Furthermore, the presently claimed subject matter concerns methods for preparing neutralizing anti HCV scFv antibodies associated with HCV clearance.

### TECHNOLOGICAL FIELD

The invention is in the field of therapeutics for hepatitis C infections.

### BACKGROUND ART

References considered to be relevant as background to the presently disclosed subject matter are listed below:
[1] Cashman, S. B., Marsden, B. D., and Dustin, L. B. (2014) The Humoral Immune Response to HCV: Understanding is Key to Vaccine Development. Frontiers in immunology 5, 550
[2] Dustin, L. B., Cashman, S. B., and Laidlaw, S. M. (2014) Immune control and failure in HCV infection—tipping the balance. Journal of leukocyte biology 96, 535-548
[3] Pierce, B. G., Keck, Z. Y., Lau, P., Fauvelle, C., Gowthaman, R., Baumert, T. F., Fuerst, T. R., Mariuzza, R. A., and Foung, S. K. (2016) Global mapping of antibody recognition of the hepatitis C virus E2 glycoprotein: Implications for vaccine design. Proceedings of the National Academy of Sciences of the United States of America
[4] Osburn, W. O., Snider, A. E., Wells, B. L., Latanich, R., Bailey, J. R., Thomas, D. L., Cox, A. L., and Ray, S. C. (2014) Clearance of Hepatitis C Infection Is Associated With the Early Appearance of Broad Neutralizing Antibody Responses. Hepatology (Baltimore, Md. 59, 2140-2151
[5] Ndongo, N., Berthillon, P., Pradat, P., Vieux, C., Bordes, I., Berby, F., Maynard, M., Zoulim, F., Trepo, C., and Petit, M. A. (2010) Association of anti-E1E2 antibodies with spontaneous recovery or sustained viral response to therapy in patients infected with hepatitis C virus. Hepatology (Baltimore, Md. 52, 1531-1542
[6] Law, M., Maruyama, T., Lewis, J., Giang, E., Tarr, A. W., Stamataki, Z., Gastaminza, P., Chisari, F. V., Jones, I. M., Fox, R. I., Ball, J. K., McKeating, J. A., Kneteman, N. M., and Burton, D. R. (2008) Broadly neutralizing antibodies protect against hepatitis C virus quasispecies challenge. Nature medicine 14, 25-27
[7] Merat, S. J., Molenkamp, R., Wagner, K., Koekkoek, S. M., van de Berg, D., Yasuda, E., Bohne, M., Claassen, Y. B., Grady, B. P., Prins, M., Bakker, A. Q., de Jong, M. D., Spits, H., Schinkel, J., and Beaumont, T. (2016) Hepatitis C virus Broadly Neutralizing Monoclonal Antibodies Isolated 25 Years after Spontaneous Clearance. PloS one 11, e0165047
[8] Bailey, J. R., Flyak, A. I., Cohen, V. J., Li, H., Wasilewski, L. N., Snider, A. E., Wang, S., Learn, G. H., Kose, N., Loerinc, L., Lampley, R., Cox, A. L., Pfaff, J. M., Doranz, B. J., Shaw, G. M., Ray, S. C., and Crowe, J. E., Jr. (2017) Broadly neutralizing antibodies with few somatic mutations and hepatitis C virus clearance. JCI insight 2 Acknowledgement of the above references herein is not to be inferred as meaning that these are in any way relevant to the patentability of the presently disclosed subject matter.

### BACKGROUND

Hepatitis C virus (HCV) is a major public health concern, with over 70 million people infected worldwide, and who are at risk for developing life-threatening liver disease. HCV infection can lead to hepatitis, cirrhosis, liver failure, and hepatocellular carcinoma (HCC); it is the leading cause of liver transplantation. HCC is the fifth most common cancer, and the third leading cause of cancer-related death worldwide (Parkin, D. M. (2006) Journal international du cancer 118, 3030-3044). No vaccine is available, and immunity against the virus is not well understood. Cure rates are expected to increase with the recent approval of Direct-Acting Antiviral Drugs (DAAs); yet despite this progress, many challenges remain, such as limited implementation, efficacy, and protection from reinfection (Hayes, C. N., and Chayama, K. (2017) Expert review of clinical pharmacology 10, 583-594). Thus, global eradication of HCV by implementing DAAs is currently not a feasible goal. Since vaccination is considered the most effective means of eradicating viral infections (Walker, C. M., and Grakoui, A. (2015) Current opinion in immunology 35, 137-143), a prophylactic HCV vaccine is an urgent, unmet medical need. However, critical gaps in understanding the correlates of protective HCV immunity have hindered the design of anti-HCV vaccines and novel immunotherapeutics.

Unlike HIV-infections, which are not spontaneously cleared, 20-40% of HCV-infected individuals experience spontaneous recovery (Thomas, D. L., et al. (2009) Nature 461, 798-801). It is now widely accepted that neutralizing antibodies (nAbs) also play a key role in viral clearance [1-3]. This point was strengthened by demonstrating that natural clearance correlates with the early development of nAbs [4], and with nAbs that exhibit distinct epitope specificity [5]. Extensive characterization of monoclonal HCV-neutralizing antibodies (mnAbs), combined with crystal structures of the HCV envelope protein E2, which is the target of most HCV-nAbs, has provided valuable information regarding the E2 antigenic landscape (Freedman, H., et al (2016) ACS infectious diseases 2, 749-762). However, since most HCV mnAbs characterized to date were generated from Chronically Infected (CI) patients [3], the nature and epitope specificities of mnAbs in Spontaneous Clearer (SC) individuals remain to be elucidated. Recent studies have demonstrated that the early appearance of broadly nAbs (bnAbs) is associated with spontaneous clearance [4]. Interestingly, bnAbs also protect against HCV infection in animal models [6]. Very recently, the first panels of bnAbs isolated from SC infections have been developed [7-8]. The panel reported by Bailey et al. displayed a low number of somatic mutations compared with the well-characterized nAbs from chronic patients exhibiting higher neutralization breadth, but were similar to nAbs from chronic infections in terms of clonality and epitope specificities [8]. It remains unknown whether and how the immune response of SC individuals is distinct from that of CI patients.

Comparing the features of antibody repertoires between distinct patient populations may provide information that can be correlated with clinically relevant outcomes. Indeed, recent studies have found common antibody sequences in unrelated individuals following Dengue (Parameswaran, P., et al. (2013) Cell host & microbe 13, 691-700), influenza (Pappas, L., et al. (2014) Nature 516, 418-422), and HIV infections (Sok, D., et al. (2013) PLoS pathogens 9, e1003754)), as well as autoimmune diseases such as celiac (Di Niro, et al. (2012) Nature medicine 18, 441-445) and pemphigus vulgaris (Cho, M. J., et al. (2014) Nature communications 5, 4167) as well as in chronic lymphocytic leukemia (Agathangelidis, A et al. (2012) Blood 119, 4467-4475).

### GENERAL DESCRIPTION

In one of its aspects, the present invention provides an isolated monoclonal antibody or any antigen-binding fragment thereof which binds to hepatitis C virus E2 protein (HCV E2), wherein said antibody is selected from a group consisting of:

a. a monoclonal antibody comprising a heavy chain complementarity determining region (CDRH) 1 denoted by SEQ ID NO. 158, CDRH2 denoted by SEQ ID NO. 159, CDRH3 denoted by SEQ ID NO. 160, and the light chain complementarity determining region (CDRL) 1 denoted by SEQ ID NO. 163, a CDRL2 denoted by SEQ ID NO. 65, and a CDRL3 denoted by SEQ ID NO. 165, or a variant thereof;

b. a monoclonal antibody comprising a CDRH1 denoted by SEQ ID NO. 168, CDRH2 denoted by SEQ ID NO. 169, CDRH3 denoted by SEQ ID NO. 170, and a CDRL1 denoted by SEQ ID NO. 173, a CDRL2 denoted by SEQ ID NO. 65, and a CDRL3 denoted by SEQ ID NO. 175, or a variant thereof;

c. a monoclonal antibody comprising a CDRH1 denoted by SEQ ID NO. 108, CDRH2 denoted by SEQ ID NO. 109, CDRH3 denoted by SEQ ID NO. 110, and a CDRL1 denoted by SEQ ID NO. 113, a CDRL2 denoted by SEQ ID NO. 65, and a CDRL3 denoted by SEQ ID NO. 115, or a variant thereof;

d. a monoclonal antibody comprising a CDRH1 denoted by SEQ ID NO. 78, CDRH2 denoted by SEQ ID NO. 79, CDRH3 denoted by SEQ ID NO. 80, and a CDRL1 denoted by SEQ ID NO. 83, a CDRL2 denoted by SEQ ID NO. 65, and a CDRL3 denoted by SEQ ID NO. 85, or a variant thereof;

e. a monoclonal antibody comprising a CDRH1 denoted by SEQ ID NO. 59, CDRH2 denoted by SEQ ID NO. 60, CDRH3 denoted by SEQ ID NO. 61, and a CDRL1 denoted by SEQ ID NO. 64, a CDRL2 denoted by SEQ ID NO. 65, and a CDRL3 denoted by SEQ ID NO. 66, or a variant thereof;

f. a monoclonal antibody comprising a CDRH1 denoted by SEQ ID NO. 69, CDRH2 denoted by SEQ ID NO. 70, CDRH3 denoted by SEQ ID NO. 182, and a CDRL1 denoted by SEQ ID NO. 73, a CDRL2 denoted by SEQ ID NO. 74, and a CDRL3 denoted by SEQ ID NO. 75, or a variant thereof;

g. a monoclonal antibody comprising a CDRH1 denoted by SEQ ID NO. 88, CDRH2 denoted by SEQ ID NO. 89, CDRH3 denoted by SEQ ID NO. 90, and a CDRL1 denoted by SEQ ID NO. 93, a CDRL2 denoted by SEQ ID NO. 65, and a CDRL3 denoted by SEQ ID NO. 95, or a variant thereof;

h. a monoclonal antibody comprising a CDRH1 denoted by SEQ ID NO. 98, CDRH2 denoted by SEQ ID NO. 99, CDRH3 denoted by SEQ ID NO. 100, and a CDRL1 denoted by SEQ ID NO. 103, a CDRL2 denoted by SEQ ID NO. 104, and a CDRL3 denoted by SEQ ID NO. 105, or a variant thereof;

i. a monoclonal antibody comprising a CDRH1 denoted by SEQ ID NO. 118, CDRH2 denoted by SEQ ID NO. 119, CDRH3 denoted by SEQ ID NO. 120, and a CDRL1 denoted by SEQ ID NO. 123, a CDRL2 denoted by SEQ ID NO. 124, and a CDRL3 denoted by SEQ ID NO. 125, or a variant thereof;

j. a monoclonal antibody comprising a CDRH1 denoted by SEQ ID NO. 128, CDRH2 denoted by SEQ ID NO. 129, CDRH3 denoted by SEQ ID NO. 130, and a CDRL1 denoted by SEQ ID NO. 133, a CDRL2 denoted by SEQ ID NO. 134, and a CDRL3 denoted by SEQ ID NO. 135, or a variant thereof;

k. a monoclonal antibody comprising a CDRH1 denoted by SEQ ID NO. 138, CDRH2 denoted by SEQ ID NO. 139, CDRH3 denoted by SEQ ID NO. 140, and a CDRL1 denoted by SEQ ID NO. 143, a CDRL2 denoted by SEQ ID NO. 65, and a CDRL3 denoted by SEQ ID NO. 145, or a variant thereof; and

l. a monoclonal antibody comprising a CDRH1 denoted by SEQ ID NO. 148, CDRH2 denoted by SEQ ID NO. 149, CDRH3 denoted by SEQ ID NO. 150, and a CDRL1 denoted by SEQ ID NO. 153, a CDRL2 denoted by SEQ ID NO. 104, and a CDRL3 denoted by SEQ ID NO. 155, or a variant thereof.

In certain embodiments, the invention provides an isolated monoclonal antibody or any antigen-binding fragment thereof which binds to HCV E2, wherein said antibody comprises a heavy chain variable region and a light chain variable region, wherein said heavy chain variable region is encoded by a nucleic acid sequence which is at least 70% identical to the nucleic acid sequence denoted by SEQ ID NO. 156, SEQ ID NO. 166, SEQ ID NO. 106, SEQ ID NO. 76, SEQ ID NO. 57, SEQ ID NO. 67, SEQ ID NO. 86, SEQ ID NO. 96, SEQ ID NO. 116, SEQ ID NO. 126, SEQ ID NO. 136, SEQ ID NO. 146, and wherein said light chain variable region is encoded by a nucleic acid sequence which is at least 70% identical to SEQ ID NO. 161, SEQ ID NO. 171, SEQ ID NO. 111, SEQ ID NO. 81, SEQ ID NO. 62, SEQ ID NO. 71, SEQ ID NO. 91, SEQ ID NO. 101, SEQ ID NO. 121, SEQ ID NO. 131, SEQ ID NO. 141, SEQ ID NO. 151.

In certain embodiments, the isolated monoclonal antibody comprises a heavy chain variable region comprising the amino acid sequence denoted by SEQ ID NO. 157, SEQ ID NO. 167, SEQ ID NO. 107, SEQ ID NO. 77, SEQ ID NO. 58, SEQ ID NO. 68, SEQ ID NO. 87, SEQ ID NO. 97, SEQ ID NO. 117, SEQ ID NO. 127, SEQ ID NO. 137, SEQ ID NO. 147, or a variant thereof and a light chain variable region comprising the amino acid sequence denoted by SEQ ID NO. 162, SEQ ID NO. 172, SEQ ID NO. 112, SEQ ID NO. 82, SEQ ID NO. 63, SEQ ID NO. 72, SEQ ID NO. 92, SEQ ID NO. 102, SEQ ID NO. 122, SEQ ID NO. 132, SEQ ID NO. 142, SEQ ID NO. 152, or a variant thereof.

In certain embodiments, the isolated monoclonal antibody is selected from a group consisting of:

a. a monoclonal antibody comprising a heavy chain variable region comprising the amino acid sequence denoted by SEQ ID NO. 157 or a variant thereof and a light chain variable region comprising the amino acid sequence denoted by SEQ ID NO. 162 or a variant thereof; and

b. a monoclonal antibody comprising a heavy chain variable region comprising the amino acid sequence denoted by SEQ ID NO. 167 or a variant thereof and a light chain variable region comprising the amino acid sequence denoted by SEQ ID NO. 172 or a variant thereof.

In certain embodiments, the isolated monoclonal antibody is a human antibody.

In certain embodiments the antibody of the invention is associated with cleared HCV infections and/or is an HCV neutralizing antibody.

In certain embodiments, the invention provides an isolated nucleic acid molecule comprising a nucleotide sequence encoding an antibody or any antigen-binding fragment thereof of the invention, as well as an expression vector comprising the isolated nucleic acid molecule and a host cell transfected with the expression vector.

In certain embodiments, the invention also provides an immunoconjugate comprising the antibody or any antigen-binding fragment thereof of the invention and an additional anti-HCV agent.

In certain embodiments, the invention also provides a pharmaceutical composition comprising as an active ingredient the isolated monoclonal antibody or any antigen-binding fragment thereof of the invention and a pharmaceutically acceptable carrier, excipient or diluent.

In certain embodiments, the pharmaceutical composition further comprises an additional anti-HCV agent.

In some embodiments, the invention provides an isolated monoclonal antibody or any antigen-binding fragment thereof which binds to HCV E2, an immunogonjugate or pharmaceutical composition comprising the same for use in a method of prophylaxis, treatment or amelioration of HCV infection.

In another aspect, the present invention provides a method of prophylaxis, treatment or amelioration of HCV infection comprising administering to a subject in need thereof a therapeutically effective amount of the isolated monoclonal antibody or any antigen-binding fragment thereof of the invention.

In certain embodiments, the method further comprises administering to the subject in need thereof an additional anti-HCV agent.

In certain embodiments, said antibody is administered at a therapeutically effective amount of 10-1000 μg/kg.

In certain embodiments, the isolated monoclonal antibody or any antigen-binding fragment thereof of the invention is for use in a method of prophylaxis, treatment or amelioration of HCV infection.

In another aspect, the present invention provides a method of detecting HCV in a biological sample obtained from a subject, said method comprising:

(a) contacting said biological sample with the isolated monoclonal antibody or any antigen-binding fragment thereof of the invention, wherein said monoclonal antibody is labeled with a detectable marker; and

(b) detecting said isolated monoclonal antibody or any antigen-binding fragment thereof; wherein the presence of said isolated monoclonal antibody or any antigen-binding fragment thereof indicates the presence of HCV in said biological sample.

The invention also provides a kit for detecting HCV comprising:

(a) at least one labeled isolated monoclonal antibody or any antigen-binding fragment thereof of the invention;

(b) means for detection of said labeled isolated monoclonal antibody; and optionally

(c) instructions for use of said kit.

In another aspect, the invention also provides a method of preparing neutralizing anti HCV scFv antibodies associated with HCV clearance, comprising the steps of:

a. constructing a phage display scFv antibody library from PBMCs obtained from SC individuals;

b. identifying at least one HCV E2 binder;

c. comparing the sequence of the at least one HCV E2 binder with the sequences in stratifying clusters from the general repertoire that are associated with HCV clearance;

d. selecting an scFv sequence with high similarity to a sequence in a stratifying cluster from the general repertoire that is associated with HCV clearance.

In certain embodiments, the scFv library construction comprises the steps of:

a. amplification of the VH and VL genes separately;

b. combinatorial assembly of the VH and VL genes; and

c. cloning into a phagemid vector.

In certain embodiments, the identification of the at least one HCV E2 binder comprises screening for phages that bind to rE2.

In certain embodiments, the stratifying clusters are selected from the clusters denoted in Tables 3 and 4.

In certain embodiments, the method further comprises the step of converting the scFv to full-length antibodies.

In certain embodiments, the full-length antibodies comprise a light chain of the selected scFv antibody and a heavy chain of one of the sequences that show high similarity to the scFv heavy chain from the general repertoire.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1C: Enrichment of the HCV-specific B-cell population in vitro. For the in vitro proliferation of B cells, CD19+ cells were isolated from PBMCs of healthy donors using a FACS sorter. Isolated B cells were labeled with CFSE, cultured in the presence of IL2, IL21, and feeder irradiated 3T3-msCD40L cells, and activated with a pool of positive peptides for 8 days.

FIG. 1A: CFSE profile of CD19+ B cells. CFSE fading (right panel) indicates the proliferation of the activated culture, compared with the non-activated culture (left panel).

FIG. 1B: Evaluating the proliferation of memory B cells. In the activated culture, 23% of the population consists of memory B cells that are positive for CD27+(right panel), compared with very low numbers of CD27+ cells in the non-activated culture (left panel).

FIG. 1C: Evaluating the ability of B cells to differentiate and produce IgG. The concentrations of IgG secreted to the culture medium 3 or 8 days following B-cell activation were measured by ELISA. (**P<0.003, ***P<0.0003, ****P<0.00003). Presented are means±SD from three independent experiments.

FIG. 2: Scheme of workflow.

The workflow included the following steps: collection of blood samples from SC, CI, and healthy individuals, sequencing of total B-cell repertoires, T-cell repertoires, and HCV-specific B-cell repertoires, analysis of repertoires and identification of antibody clusters and TCR sequences associated with viral clearance, construction of an antibody phage display library, isolation of a panel of HCV-binding antibody sequences that associate with cleared infections, and integration of all data to construct HCV-broadly neutralizing antibodies associated with clearance. For AIRR sequencing (upper right panel) the step of sample processing library preparation refers to separation of HCV-specific B cells from whole repertoire (RNA extraction, Reverse transcription, PCRs to enrich antibodies and addition of UMI and adaptors). For construction of HCV-specific antibodies (lower right panel), the following steps are performed i.e. integration of phage display and repertoire data to construct HCV-specific antibodies and characterization of the antibodies. For detection of binding antibodies (lower left panel), sample processing library preparation relates to RNA extraction, Reverse transcription, PCR on VH and VL, Cloning into phage display vectors and Assembly into ScFvs. In addition, Phage display relates to Incubation with solid phase antigens, Elution of specific phage, Sequencing and expression of specific antibodies and Measurement of affinity to HCV E2.

FIG. 3A-3D: Characterization of sera from HCV-infected individuals.

FIG. 3A: HCV antibodies binding to rE2 protein (0.5 μg/ml) performed with 1:1000 diluted sera of CI (n=52) and SC (n=18) by ELISA. Each dot represents a patient. The background of the binding to BSA was subtracted from all samples. Presented are mean OD (450 nm) values from three independent experiments.

FIG. 3B: The HCVcc neutralization assays were performed with 1:1000 diluted sera of CI (n=52) and SC (n=18) to screen for antibodies that can neutralize HCV infection. The Y axis shows the percentage of neutralization capacity compared with neutralization by sera from a healthy control. Each dot represents the mean neutralization for a patient, from three independent experiments.

FIG. 3C: Characterizing HCV binding and neutralizing in sera obtained from two patients (CI21 and CI22) before and after anti-HCV treatment and following SVR by ELISA (with 0.5 μg/ml rE2 protein and 1:1000 diluted sera)

FIG. 3D: Characterizing HCV binding and neutralizing in sera obtained from two patients (CI21 and CI22) before and after anti-HCV treatment by the HCVcc neutralization assay (with 1:1000 diluted sera).

The HCV-cured blood samples were collected from six months to one year after achieving a sustained viral response. **P<0.003, ***P<0.0003. Presented are means±SD from three independent experiments.

FIG. 4A-4E: Characterization of B-cell repertoires in SC, CI, and healthy individuals.

FIG. 4A: The number of unique sequences per sample after pre-processing.

FIG. 4B: The CDR3 length distribution.

FIG. 4C: The IGHV gene distribution. Only functional V genes that were in the 15 topmost frequent in at least one sample are shown.

FIG. 4D: The IGHJ gene distribution.

FIG. 4E: Feature combinations whose abundance differ between the SC and CI groups are presented for sequence clusters grouped by identical IGHV and IGHJ and by high CDR3 similarity, which were significantly more abundant in either SC or CI cohort (I #samplesSC—#samplesCII>3 samples). Sequence logos CDR3 of these clusters are presented in FIG. 6.

FIG. 5A-5D: General characterization of T-cell repertoires of resolved and chronic HCV infection.

FIG. 5A: The number of sequences per sample after pre-processing.

FIG. 5B: TRBJ gene usage, in each clinical group.

FIG. 5C: CDR3 length distribution per sample, in each clinical group.

FIG. 5D: TRBV gene usage, in each clinical group.

FIG. 6. CDR3 from the SC and CI abundant B cells clusters.

Sequence logos of the overall nucleic acid composition of the CDR3s in copious clusters. The individual abundance of these clusters is shown in FIG. 4E. The nucleic acid sequences appearing in the figure at the left side from the top to the bottom are as denoted by SEQ ID NO: 84, 94, 114, 164, 174, 154 and 187 and at the right side, from the top to the bottom are as denoted by SEQ ID NO: 188, 189, 190, 191, 192 and 193.

FIG. 7A-7D: IGHV mutation characterization in SC and CI infections. FIG. 7A: Isotype usage distribution.

FIG. 7B: IGHV mutation distribution, per isotype.

FIG. 7C: IGHV mutation distribution per isotype per cohort.

FIG. 7D: IGHV mutation distribution per isotype per cohort per IGHV gene.

Only statistically significant combinations are shown (P<0.05, t-test).

FIG. 8A-8D: Machine learning model used to stratify between SC and CI.

FIG. 8A: Accuracy was based on the B cells' repertoire. Original labels represent clustered sequences by identical IGHV and IGHJ and the high similarity of the CDR3 amino acid sequence. For validation purposes, the model was trained and applied on randomly labeled data.

FIG. 8B: Prediction model based on the T cells' repertoire. The training for the T-cell repertoires model is very similar to the B-cell model, except that the data were clustered solely by CDR3 amino acid identity.

FIG. 8C: The top 10 clusters used by the model to stratify between the cohorts in B-cell clusters.

Sequence logos of the CDR3 of the B cell clusters are presented in FIG. 9.

FIG. 8D: The top 10 clusters used by the model to stratify between the cohorts in T-cell clusters. The amino acid sequences of the CDR3 are as denoted by 144, 194, 195, 196, 197, 198, 199, 200, 201 and 184 (from left to right).

FIG. 9: CDR3 from the SC and CI B cells clusters used for the Logistic Regression model.

Sequence logos of the overall AA composition across the CDR3s in the top 10 clusters used by the model to stratify between the cohorts. The individual abundance of these clusters is shown in FIG. 8C. The sequences appearing in the figure at the left side, from the top to the bottom are as denoted by SEQ ID NO: 202, 203, 204, 205 and 206 and at the right side, from the top to the bottom are as denoted by SEQ ID NO: 207, 208, 209, 210 and 211.

FIG. 10A-10E: Isolation of HCV-specific B cells from resolved and chronic HCV infection.

FIG. 10A: HCV-specific B-cells isolated from six CI and three SC individuals, as compared with control healthy individuals. The fold enrichment of HCV-specific B cells from each sample was calculated compared with the number of B cells isolated from a healthy individual, as demonstrated in FIG. 11.

FIG. 10B: HCVcc-neutralization assays using supernatants of cultured B cells from healthy, SC, and CI samples after two weeks of activation in vitro. (*P<0.03, **P<0.003, ***P<0.0001, ****P<0.00003, t-test).

FIG. 10C: Mutation numbers in IGHV genes in the general repertoire compared with the HCV-specific repertoire. Each specific sequence was randomly matched to a non-specific sequence with the same IGHV and IGHJ genes. The sequences were grouped by isotype and mutations were compared by Mann Whitney test (IGA p=3.488873e-07, IGG p=6.8495Ile-08, IGM p=3.764229e-04).

FIG. 10D: Mutation number in the IGHV genes in the specific repertoire for SC and CI (IGA p=0.000574, IGG p=0.435930).

FIG. 10E: Conserved amino acids in CDR3 from the HCV-specific repertoire (binders as denoted by SEQ ID NO: 185) compared with the general repertoire (non-binders as denoted by SEQ ID NO: 186). For each specific sequence, a non-specific sequence was randomly matched. Sequences were then grouped by IGHV, IGHJ, and CDR3 length. Cases where CDR3 amino acids were very conserved for binder sequences but not for non-binders are shown.

FIG. 11: Isolation of HCV-specific B cells from SC, CI, and healthy donors by FACS.

CD19+ B cells from SC17 and CI58 were grown with feeder-irradiated 3T3-msCD40L cells and activated with 5 μg/ml rE2 protein, IL2, and IL21 for 13-14 days. After 14 days, activated B cells were incubated with 5 μg/ml rE2 and stained with CD19-PE, CD27-BV421, and tagged rE2 (anti-cMyc, alexa fluor 633). Viable, CD19+, CD27+, and HCsAg+ were isolated by FACS. The gating region is shown as a black rectangular.

FIG. 12A-12D: Identification of HCV-specific antibody sequences associated with HCV infection clearance.

FIG. 12A: Binding of the phage-displayed antibodies to the rE2 protein (5 μg/ml) by ELISA. Each bar indicates the mean fold change ±SD in the OD compared with BSA binding, from three independent experiments.

FIG. 12B: Violin plot of the distances between HCV-specific sequences and the healthy, CI and SC repertoires.

FIG. 12C: Phylogenetic trees of the two closest clusters to scFv SC11.

FIG. 12D: Phylogenetic trees of the two closest clusters to scFv SC28.

FIG. 13 is a scheme showing sequence alignment of the isolated antibodies. The sequences of the VH of the antibodies (top alignment) are as denoted by SEQ ID NO: 58, 147, 117, 97, 87, 127, 137, 68, 77, 167, 107 and 157 (from the bottom to the top). The sequences of the VL of the antibodies (bottom alignment) are as denoted by SEQ ID NO: 63, 82, 172, 92, 112, 162, 142, 102, 152, 72, 132 and 122.

FIG. 14A is a graph showing specific binding of Abs to rE2 in an ELISA. ELISA plate was coated with rE2. Binding assays were performed with purified antibodies; CI 16, 81 and 92; SC 1, 3, and 17. Binding was detected with goat anti human conjugated to HRP.

FIG. 14B: Specific binding of the antibodies to rE2 compared to BSA in an ELISA.

FIG. 14C-14I are graphs showing antibody neutralization assays. Huh7.5 cells were incubated with the virus and antibodies, CI 16, 81 and 92; SC 1, 3, 17, and 93 (C-I respectively) or irrelevant antibody. Neutralization was measured by immunofluorescence microscopy and manual counting of foci stained by human pAb.

FIG. 15: The distance between scFv antibody sequences and clusters from B-cell repertoires of SC and CI infection.

Each dot represents the average distances between the scFv antibody sequence and the 10 closest sequences (by VDJ, amino acid sequence) of the B-cell repertoire from healthy controls (C), CI, and SC. The lower the distance, the more similar is the scFv antibody sequence.

FIG. 16A-16D: Construction and characterization of antibodies correlated with infection clearance.

FIG. 16A: Binding of antibodies RMS28 and RMS11 to the rE2 protein (5 μg/ml) compared with the phage display antibodies SC28 and SC11 by ELISA, using 16 μg/ml Ab. Each bar indicates the mean fold change ±SD in binding, compared with BSA, from three independent experiments.

FIG. 16B: Binding of antibodies RMS11 and RMS28 to the rE2 protein (5 μg/ml), compared with a well-defined panel of nAbs and a nonspecific control antibody RO4 by ELISA, using 16 μg/ml Ab. Presented are mean OD (450 nm) values ±SD, from three independent experiments.

FIG. 16C: HCVcc neutralization assays were carried out with genotypes G1-G7 using 20 μg/ml of antibody RMS11 (SC11).

FIG. 16D: HCVcc neutralization assays were carried out with genotypes G1-G7 using 20 μg/ml of antibody RMS28 (SC28).

The percent neutralization was calculated as the percent reduction in FFU compared with virus incubated with an irrelevant control antibody (R04). Presented are means of % neutralization ±SD from three independent experiments.

FIG. 17: Schematic representation of the sequences of the heavy (VH) and light (VL) chains of the antibodies VH16618 (RMS11 as denoted by SEQ ID NO: 167 AND 172) and VH510520 (RMS28 as denoted by SEQ ID NO: 157 AND 162) with CDRs highlighted in grey.

FIG. 18: Schematic representation of the sequences of the heavy (VH) and light (VL) chains of the antibodies SC28 (as denoted by SEQ ID NO: 107 AND 112), SC11(as denoted by SEQ ID NO: 77 AND 82), SC1(as denoted by SEQ ID NO: 58 AND 63), SC3 (as denoted by SEQ ID NO: 68 AND 72), CI16 (as denoted by SEQ ID NO: 87 AND 92), SC17 (as denoted by SEQ ID NO: 97 AND 102), SC76 (as denoted by SEQ ID NO: 117 AND 122), CI81 (as denoted by SEQ ID NO: 127 AND 132), CI92 (as denoted by SEQ ID NO: 137 AND 142) and SC93 (as denoted by SEQ ID NO: 147 AND 152) with CDRs highlighted in grey.

### DETAILED DESCRIPTION OF EMBODIMENTS

The present invention is based on an in-depth analysis of HCV-specific immune response and the identification of features that correlate with infection outcome. The inventors compared the general B- and T-cell repertoires, as well as the HCV-specific B-cell repertoires, of spontaneous clearer (SC) of HCV infection and chronically infected (CI) individuals. The inventors demonstrated that SC individuals and CI patients develop clusters of antibodies with distinct properties that can accurately stratify between the SC and CI samples. These clusters were termed herein “stratifying clusters”. Clones that can accurately stratify between SC and CI samples were termed accordingly “stratifying clones”.

The stratifying clusters in accordance with the invention are listed in Tables 3 and 4. Strikingly, the inventors found that enrichment of specific clusters in SC or CI is indicative of infection outcome, and with an accuracy of over 90% for B-cell repertoires and 80% for T-cell repertoires. This may have important clinical relevance as well as prognostic value for the outcome of an active infection.

These unique characteristics were used in a machine learning framework to accurately predict infection outcome. Using combinatorial antibody phage display library technology, HCV-specific antibody sequences were identified. By integrating these data with the repertoire analysis, several antibodies were constructed which are characterized by high neutralization breadth, and which are associated with infection clearance.

The effective neutralizing antibodies have an important clinical implication as post-exposure prophylaxis for accidental needle-stick or other percutaneous or mucosal exposure. The second and most relevant application would be in the prevention of recurrent HCV infection in the liver which may occur within 24 hours post liver transplantation and attack the new liver. Reinfection of the transplanted liver is universal and 20% to 30% of recurrence results in accelerated progression of fibrosis that can lead to cirrhosis in 5 years. Passively administered neutralizing antibody, with or without concomitant antiviral therapy, offers a viable and promising option to suppress/eradicate HCV before it infects the naive transplanted liver, as used in the case of liver transplantation on the background of HBV infections.

The invention therefore provides a method for constructing antibodies that are correlated with successful infection clearance.

The method generally comprising:
preparing a panel of anti HCV E2 monoclonal antibodies (e.g. in a form of a phage display library);
screening the nucleic acid sequences of said antibodies to identify sequences with similarity to sequences of stratifying clusters that are associated with HCV clearance;
selecting anti HCV antibodies with high similarity to a stratifying cluster from the general repertoire that is associated with HCV clearance; and optionally
combining the light chain variable region of the selected antibody with the heavy chain variable region of said highly similar antibody from the stratifying cluster that is associated with HCV clearance.

High sequence similarity is defined herein as having 95% or higher (e.g. 96%, or 97%, or 98%, or 99% or 100%) of total sequence identity or junctions identity.

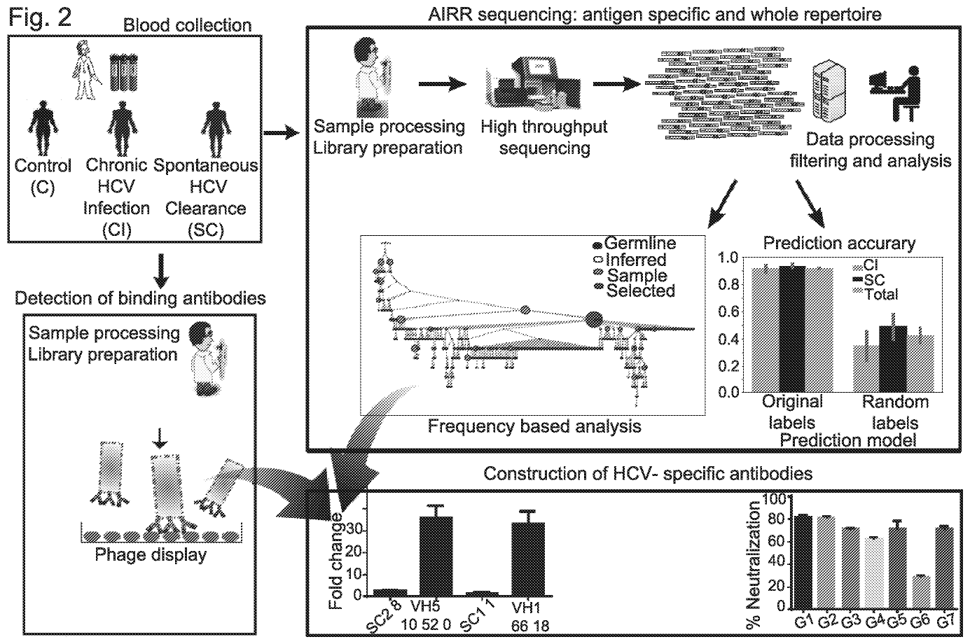

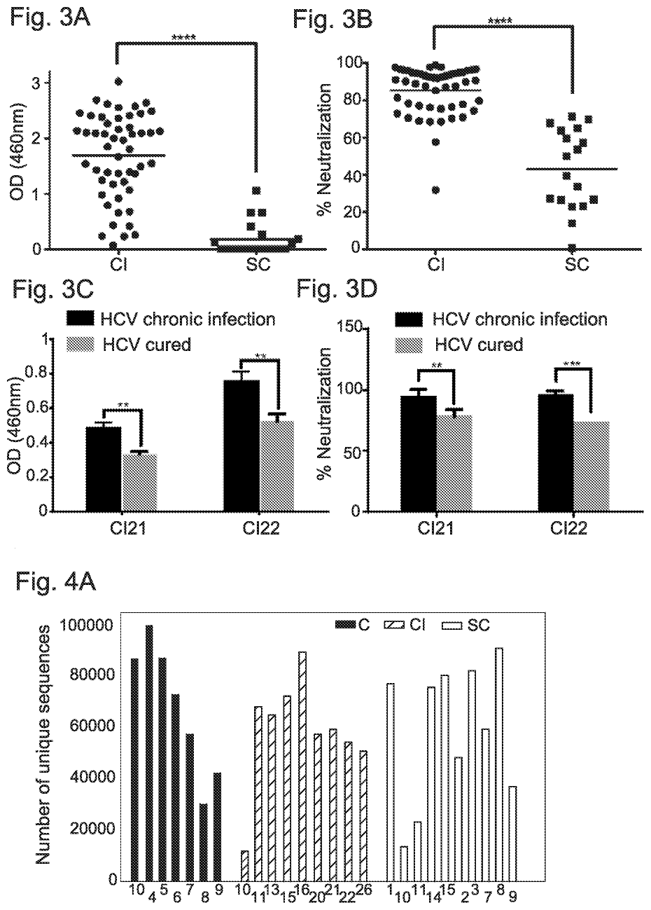

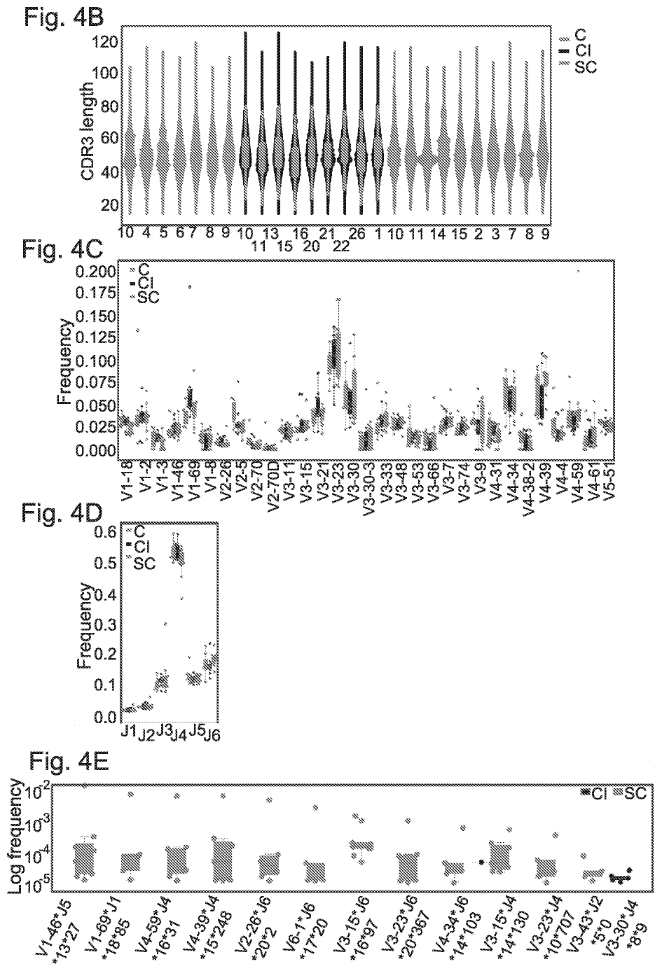

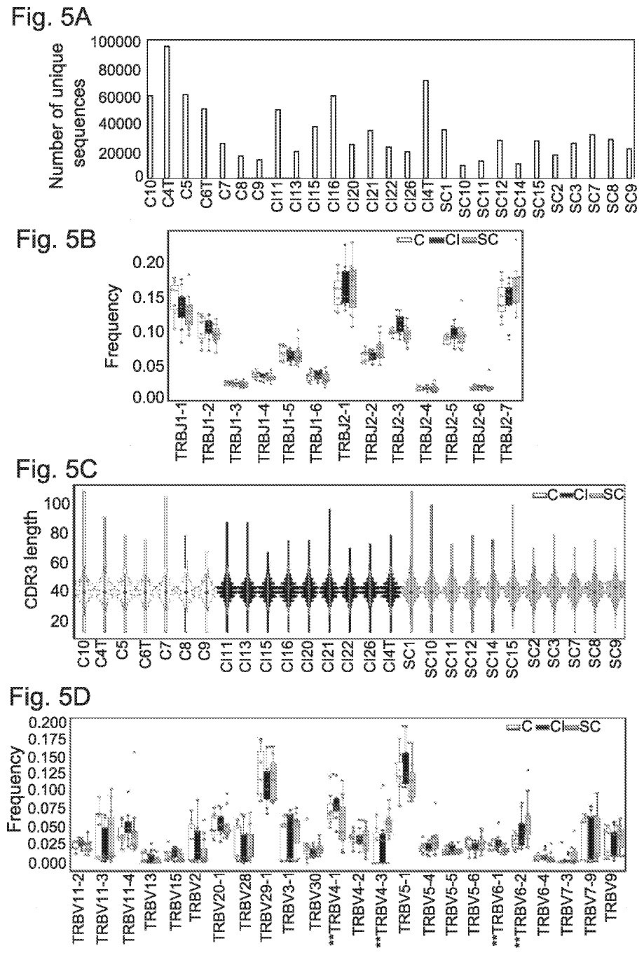

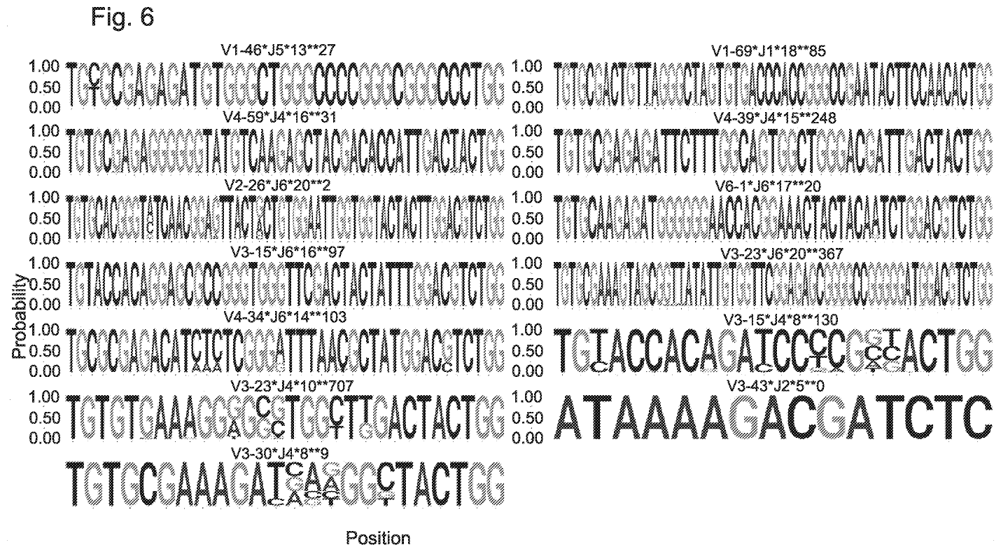

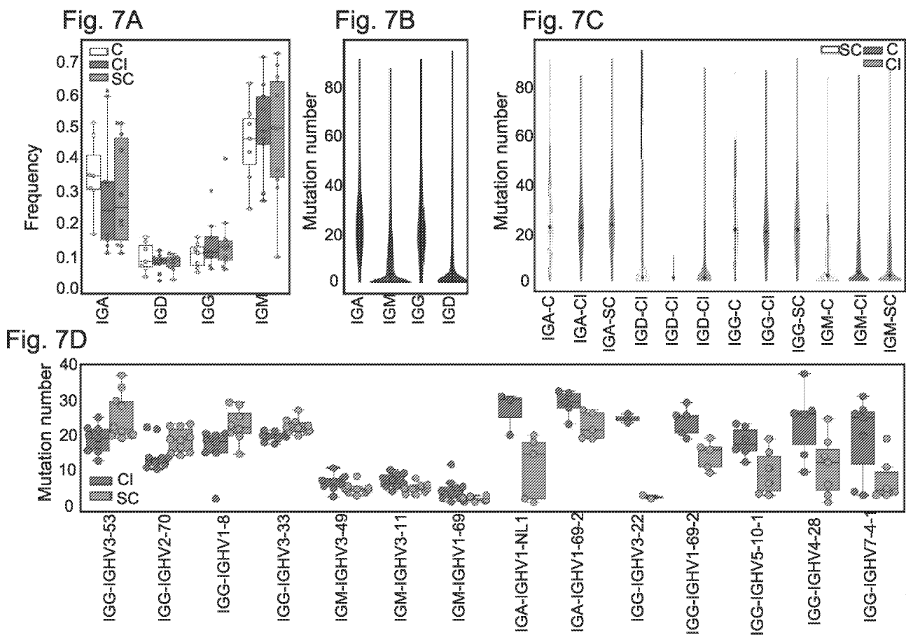

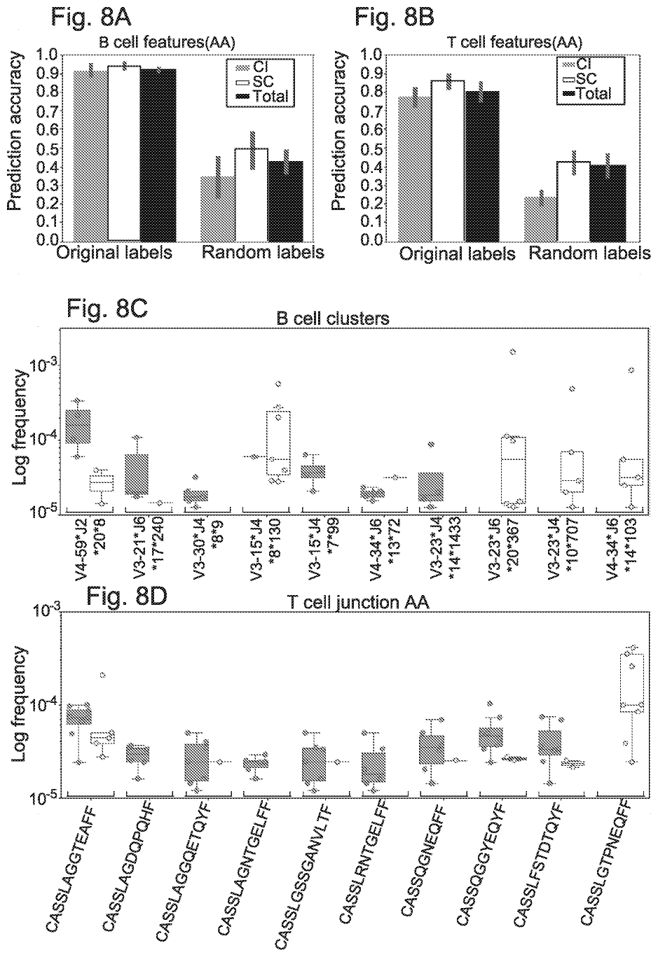

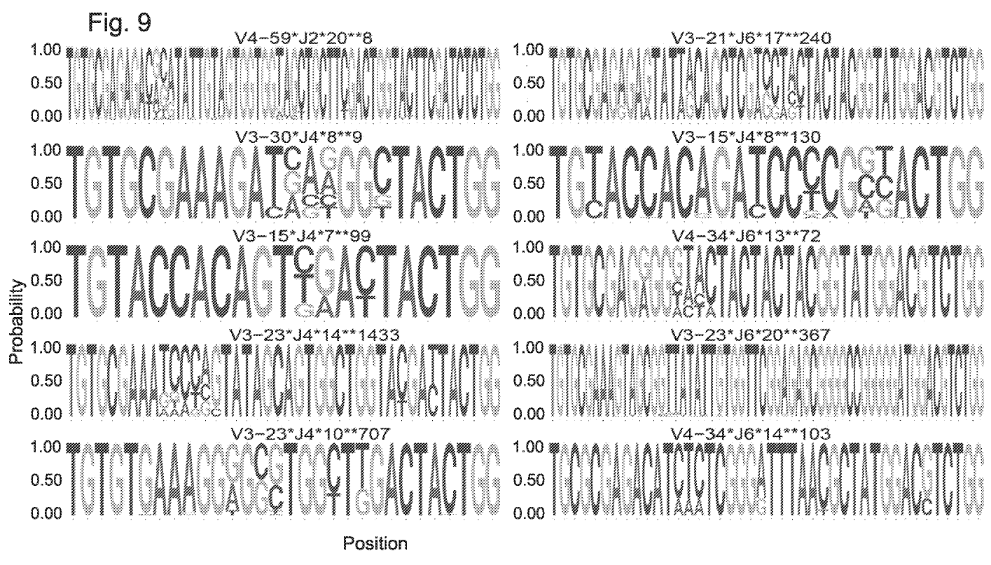

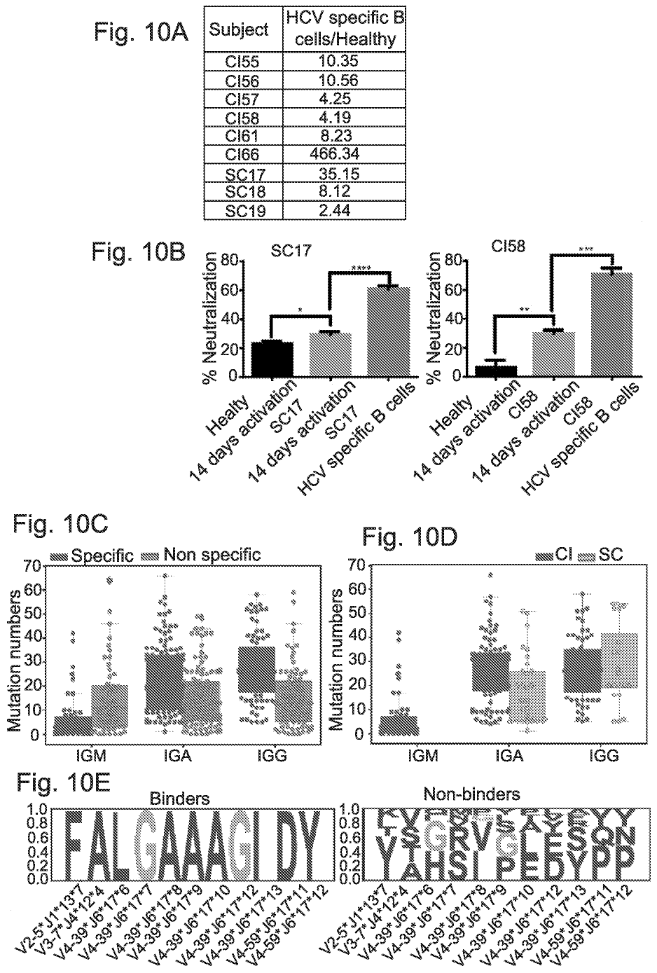

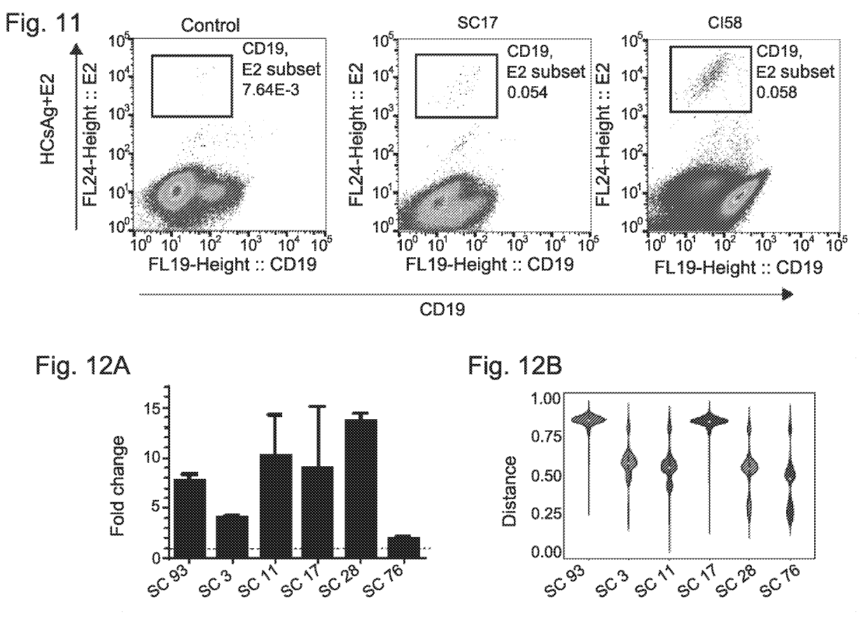

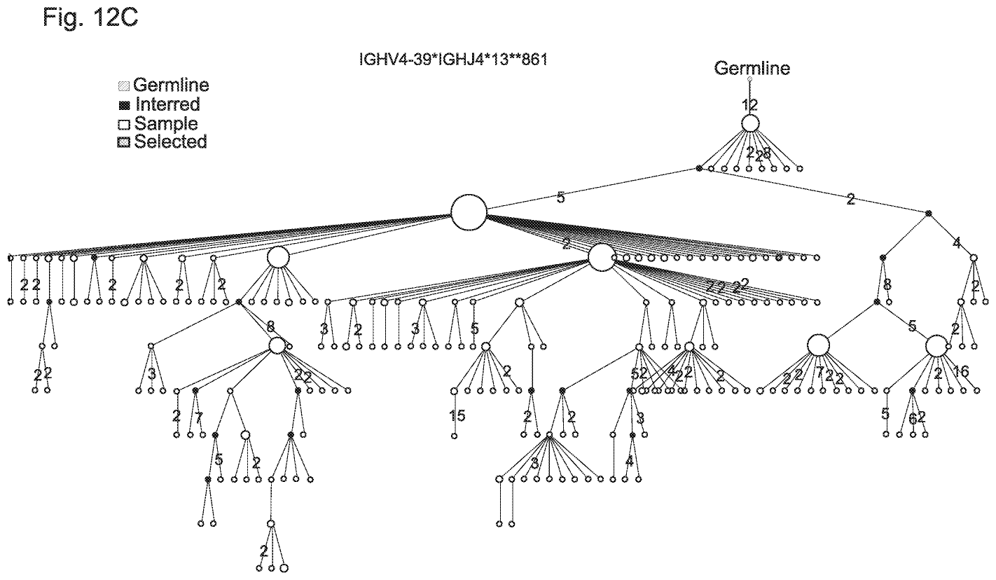

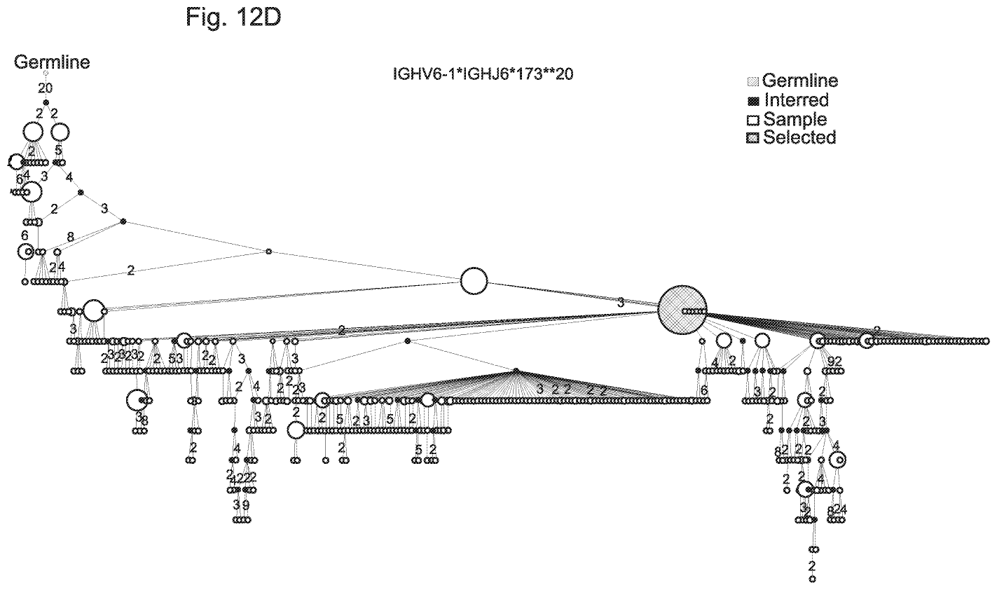

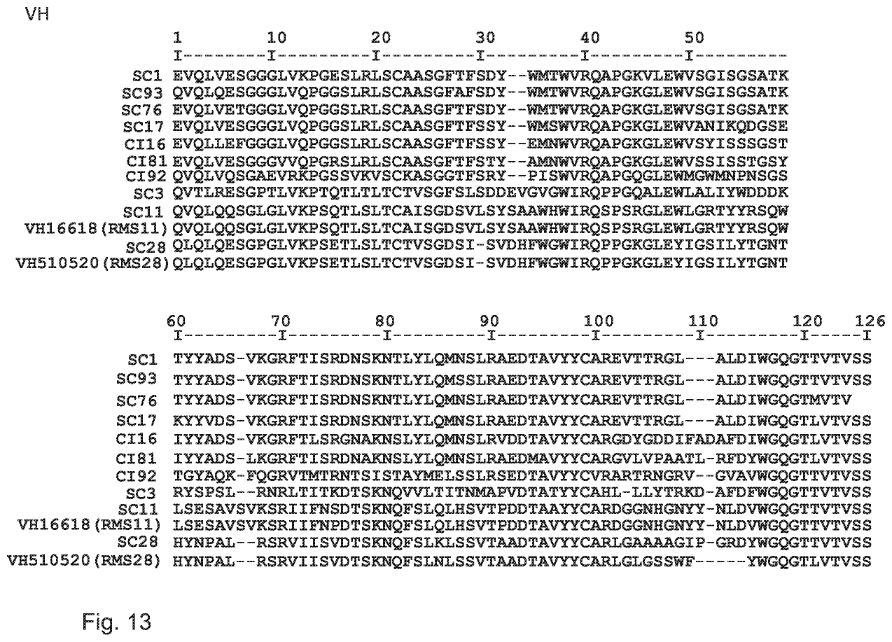

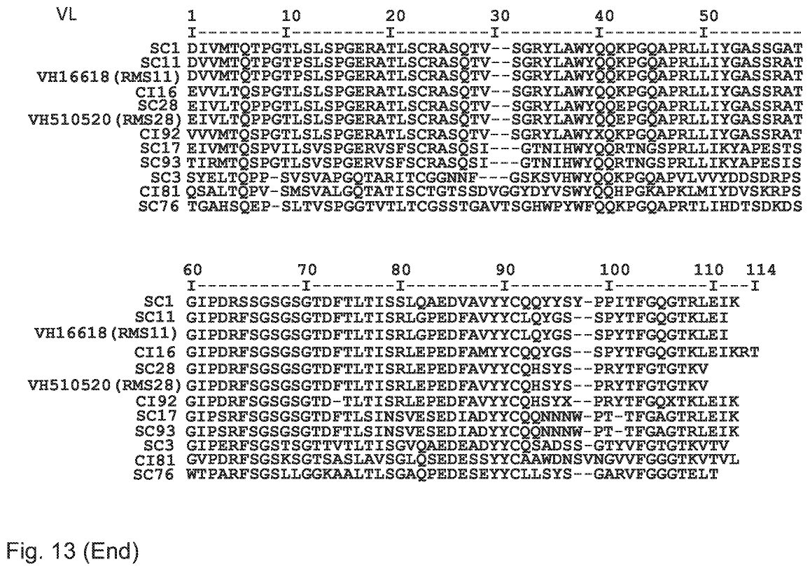

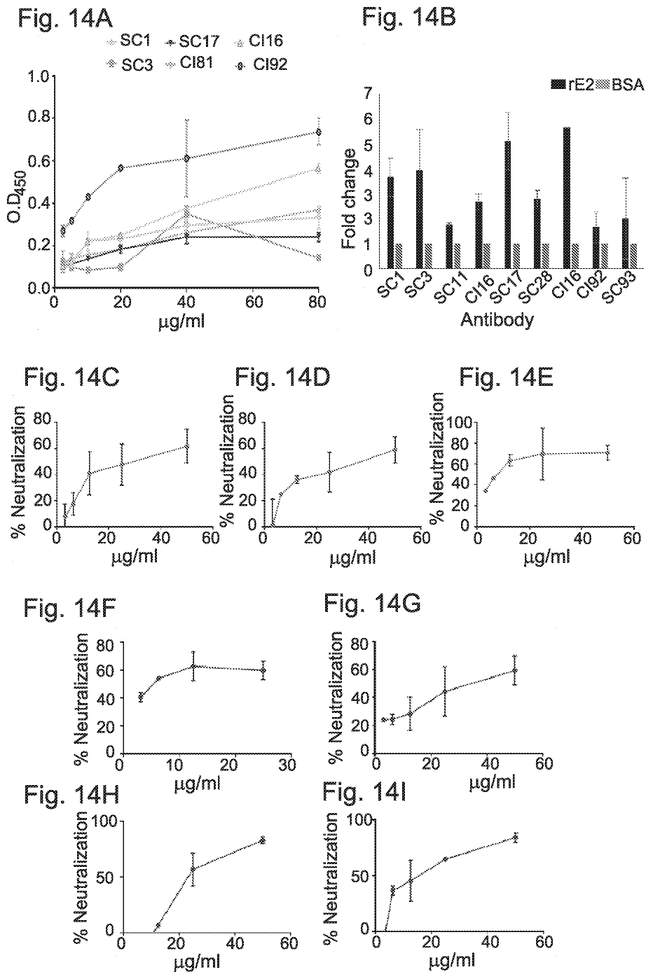

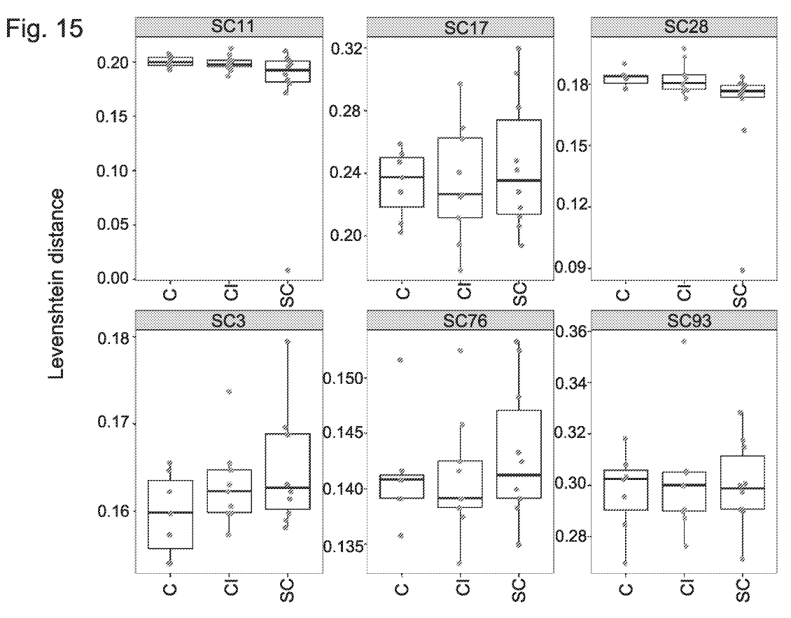

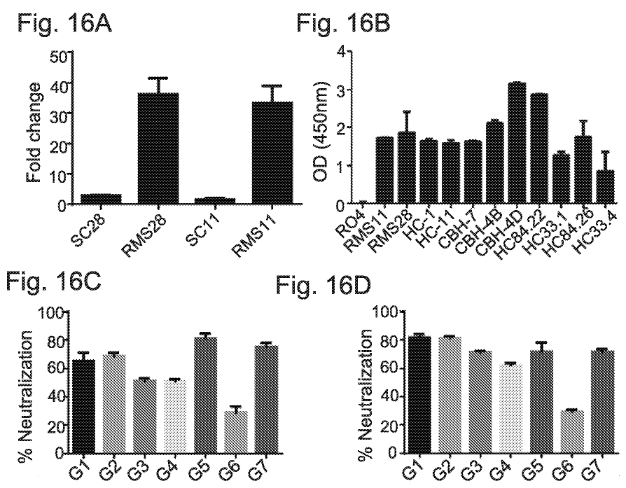

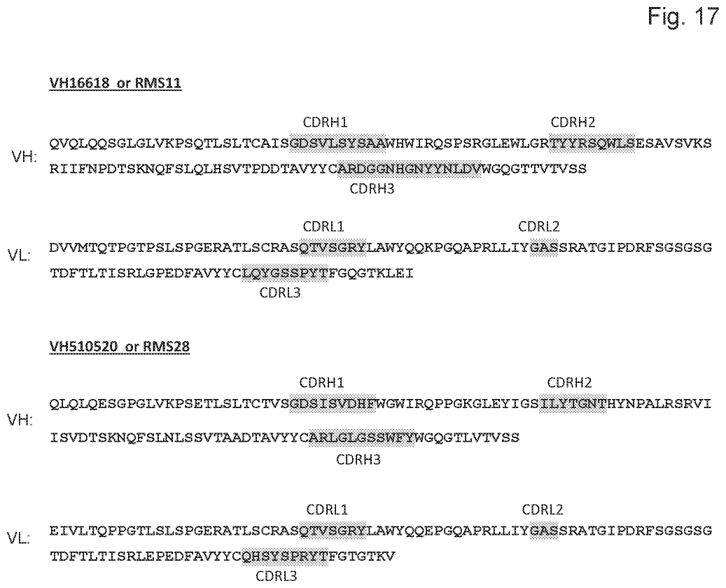

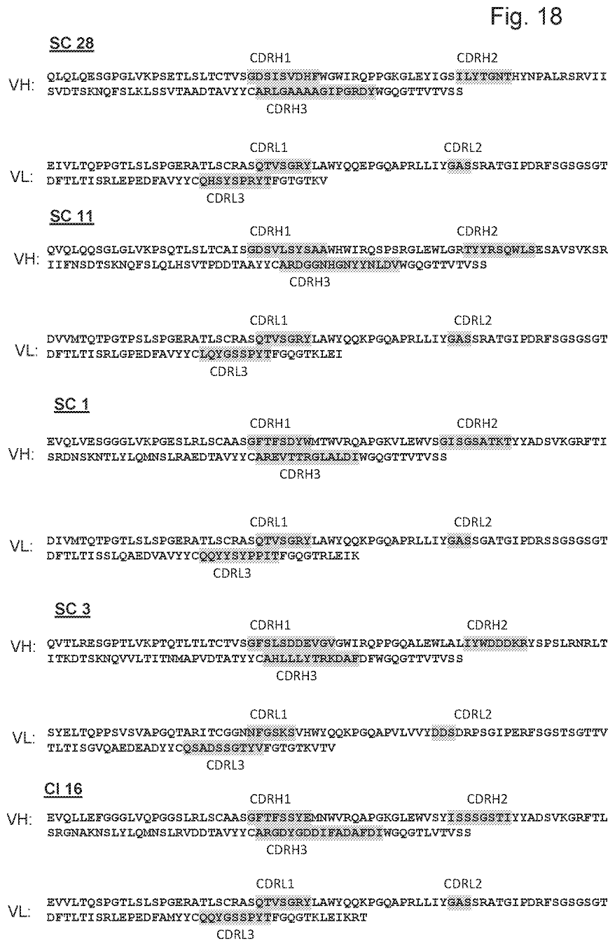

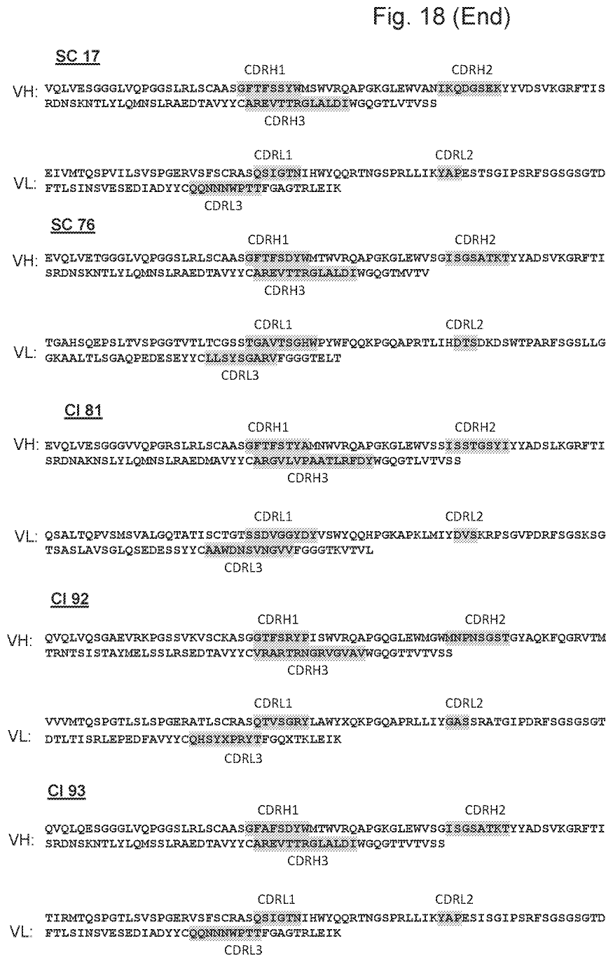

The overall approach of the invention is exemplified in FIG. 2.

The invention also provides anti HCV antibodies as will be described below, as well as pharmaceutical compositions and methods of treatment and prophylaxis using the antibodies of the invention.

The invention also provides a method of prognosis of the status of infection.

### Definitions

The term “HCV E2” refers to a structural protein found in the hepatitis C virus. It is present on the viral membrane (envelope protein) and functions as a host receptor binding protein, mediating entry into host cells. The HCV E2 protein may be prepared using any method known in the art, for example by recombinant methods, as described in the Examples below.

As indicated above, the present invention provides isolated monoclonal antibodies that bind to HCV E2. The term “antibody” refers to a polypeptide encoded by an immunoglobulin gene or functional fragments thereof that specifically binds and recognizes an antigen, namely HCV E2.

The term “monoclonal antibody”, “monoclonal antibodies” or “mAb” as herein defined refers to a population of substantially homogenous antibodies, i.e., the individual antibodies comprising the population are identical except for possibly naturally occurring mutations that may be present in minor amounts. Monoclonal antibodies are directed against a single antigenic site (epitope).

Monoclonal antibodies may be prepared and purified by any method known in the art. For example, monoclonal antibodies may be prepared from B cells taken from the spleen or lymph nodes of immunized animals (e.g. rats, mice or monkeys), by fusion with immortalized B cells under conditions which favor the growth of hybrid cells.

Alternatively, monoclonal antibodies may be prepared from peripheral blood mononuclear cells (PBMC) obtained from patients that were infected with HCV. mRNA may be isolated from these cells and used for variable heavy and variable light (VH/VL) chain amplification and further used for example for constructing a phage display library, in order to select active antibodies. Based on the results obtained from a phage display library, full length antibodies are produced, as known in the art and as described below.

The phage display libraries can be prepared from PBMC of HCV patients that are defined as spontaneously clearers (SC) or chronically infected (CI). Subjects were defined as spontaneously cleared of HCV if anti-HCV antibodies are detectable, with undetectable HCV RNA as assessed for example by the Taqman reverse-transcription polymerase chain reaction (RT-PCR) quantitative assays. Chronically infected HCV patients were defined as such if there were detectable viral loads for more than 1 year.

Purification of monoclonal antibodies may be performed using any method known in the art, for example by affinity chromatography, namely, by using an affinity column to which a specific epitope (or antigen) is conjugated. Alternatively purification of antibodies may be based on using protein A column chromatography, as described below.

An exemplary antibody structural unit comprises a tetramer, as known in the art. Each tetramer is composed of two identical pairs of polypeptide chains, each pair having one “light chain” and one “heavy chain”. The N-terminus of each chain defines a variable region of about 100 to 110 or more amino acids primarily responsible for antigen (or epitope) recognition.

Thus, the terms “heavy chain variable region” (V_{H}) and “light chain variable region” (V_{L}) refer to these heavy and light chains, respectively. More specifically, the variable region is subdivided into hypervariable and framework (FR) regions. Hypervariable regions have a high ratio of different amino acids in a given position, relative to the most common amino acid in that position. Four FR regions which have more stable amino acids sequences separate the hypervariable regions. The hypervariable regions directly contact a portion of the antigen's surface. For this reason, hypervariable regions are herein referred to as “complementarily determining regions”, or “CDRs”, the CDRs are positioned either at the heavy chain of the antibody (“a heavy chain complementarity determining region”) or at the light chain of the antibody (a “light chain complementarity determining region”).

From N-terminal to C-terminal, both light and heavy chains comprise the domains FR1, CDR1, FR2, CDR2, FR3, CDR3 and FR4. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located.

Thus, the complementarity determining regions CDRH1, CDRH2 and CDRH3 refer to the three complementarity determining regions starting from the N-terminus of the antibody's heavy chain (also referred to herein as heavy chain complementarity determining region) and the complementarity determining regions CDRL1, CDRL2 and CDRL3 refer to the three complementarity determining regions starting from the N-terminus of the antibody's light chain (also referred to herein as light chain complementarity determining region).

For example, as demonstrated in FIG. 17 in the context of the heavy chain of the antibody referred to herein as “VH16618” (also referred to herein as “RMS11”), CDRH1, CDRH2 and CDRH3 appear as grey boxes in the amino acid sequence of the heavy chain of the antibody. The respective CDRL1, CDRL2 and CDRL3 of the antibody referred to herein as VH16618, appear in FIG. 17 in the context of the light chain of the antibody.

Binding of antibodies or antigen-binding fragments thereof to HCV E2 may be determined using any method known in the art, for example using an ELISA assay as described below or BIAcore analysis.

In some embodiments the isolated monoclonal antibody according to the invention comprises the CDRH1 denoted by SEQ ID NO. 168, CDRH2 denoted by SEQ ID NO. 169, and CDRH3 denoted by SEQ ID NO. 170, and the CDRL1 denoted by SEQ ID NO. 173, CDRL2 denoted by SEQ ID NO. 65, and the CDRL3 denoted by SEQ ID NO. 175, or a variant thereof. This antibody is referred to herein as “VH16618” or “RMS11”.

In other embodiments the isolated monoclonal antibody as herein defined comprises the CDRH1 denoted by SEQ ID NO. 158, CDRH2 denoted by SEQ ID NO. 159, and the CDRH3 denoted by SEQ ID NO. 160, and the CDRL1 denoted by SEQ ID NO. 163, CDRL2 denoted by SEQ ID NO. 65, and the CDRL3 denoted by SEQ ID NO. 165, or a variant thereof. This antibody is referred to herein as “VH510520” or “RMS28”.

In other embodiments the isolated monoclonal antibody as herein defined comprises the CDRH1 denoted by SEQ ID NO. 108, CDRH2 denoted by SEQ ID NO. 109, and the CDRH3 denoted by SEQ ID NO. 110, and the CDRL1 denoted by SEQ ID NO. 113, CDRL2 denoted by SEQ ID NO. 65, and the CDRL3 denoted by SEQ ID NO. 115, or a variant thereof. This antibody is referred to herein as “SC28”.

In other embodiments the isolated monoclonal antibody as herein defined comprises the CDRH1 denoted by SEQ ID NO. 78, CDRH2 denoted by SEQ ID NO. 79, and the CDRH3 denoted by SEQ ID NO. 80, and the CDRL1 denoted by SEQ ID NO. 83, CDRL2 denoted by SEQ ID NO. 65, and the CDRL3 denoted by SEQ ID NO. 85, or a variant thereof. This antibody is referred to herein as “SC11”.

In other embodiments the isolated monoclonal antibody as herein defined comprises the CDRH1 denoted by SEQ ID NO. 59, CDRH2 denoted by SEQ ID NO. 60, and the CDRH3 denoted by SEQ ID NO. 61, and the CDRL1 denoted by SEQ ID NO. 64, CDRL2 denoted by SEQ ID NO. 65, and the CDRL3 denoted by SEQ ID NO. 66, or a variant thereof. This antibody is referred to herein as “SC1”.

In other embodiments the isolated monoclonal antibody as herein defined comprises the CDRH1 denoted by SEQ ID NO. 69, CDRH2 denoted by SEQ ID NO. 70, and the CDRH3 denoted by SEQ ID NO. 182, and the CDRL1 denoted by SEQ ID NO. 73, CDRL2 denoted by SEQ ID NO. 74, and the CDRL3 denoted by SEQ ID NO. 75, or a variant thereof. This antibody is referred to herein as “SC3”.

In other embodiments the isolated monoclonal antibody as herein defined comprises the CDRH1 denoted by SEQ ID NO. 88, CDRH2 denoted by SEQ ID NO. 89, and the CDRH3 denoted by SEQ ID NO. 90, and the CDRL1 denoted by SEQ ID NO. 93, CDRL2 denoted by SEQ ID NO. 65, and the CDRL3 denoted by SEQ ID NO. 95, or a variant thereof. This antibody is referred to herein as “CI16”.

In other embodiments the isolated monoclonal antibody as herein defined comprises the CDRH1 denoted by SEQ ID NO. 98, CDRH2 denoted by SEQ ID NO. 99, and the CDRH3 denoted by SEQ ID NO. 100, and the CDRL1 denoted by SEQ ID NO. 103, CDRL2 denoted by SEQ ID NO. 104, and the CDRL3 denoted by SEQ ID NO. 105, or a variant thereof. This antibody is referred to herein as “SC17”.

In other embodiments the isolated monoclonal antibody as herein defined comprises the CDRH1 denoted by SEQ ID NO. 118, CDRH2 denoted by SEQ ID NO. 119, and the CDRH3 denoted by SEQ ID NO. 120, and the CDRL1 denoted by SEQ ID NO. 123, CDRL2 denoted by SEQ ID NO. 124, and the CDRL3 denoted by SEQ ID NO. 125, or a variant thereof. This antibody is referred to herein as “SC76”.

In other embodiments the isolated monoclonal antibody as herein defined comprises the CDRH1 denoted by SEQ ID NO. 128, CDRH2 denoted by SEQ ID NO. 129, and the CDRH3 denoted by SEQ ID NO. 130, and the CDRL1 denoted by SEQ ID NO. 133, CDRL2 denoted by SEQ ID NO. 134, and the CDRL3 denoted by SEQ ID NO. 135, or a variant thereof. This antibody is referred to herein as “CI81”.

In other embodiments the isolated monoclonal antibody as herein defined comprises the CDRH1 denoted by SEQ ID NO. 138, CDRH2 denoted by SEQ ID NO. 139, and the CDRH3 denoted by SEQ ID NO. 140, and the CDRL1 denoted by SEQ ID NO. 143, CDRL2 denoted by SEQ ID NO. 65, and the CDRL3 denoted by SEQ ID NO. 145, or a variant thereof. This antibody is referred to herein as “CI92”.

In other embodiments the isolated monoclonal antibody as herein defined comprises the CDRH1 denoted by SEQ ID NO. 148, CDRH2 denoted by SEQ ID NO. 149, and the CDRH3 denoted by SEQ ID NO. 150, and the CDRL1 denoted by SEQ ID NO. 153, CDRL2 denoted by SEQ ID NO. 104, and the CDRL3 denoted by SEQ ID NO. 155, or a variant thereof. This antibody is referred to herein as “SC93”.

The CDRs of the antibodies referred to herein are presented in the sequence listing as well as in the context of their respective heavy and light chain sequences, e.g. in FIG. 17 and in FIG. 18.

By the term “variant” it is meant sequences of amino acids or nucleotides that are different from the sequences specifically identified herein, namely, in which one or more amino acid residues or nucleotides are deleted, substituted or added.

It should be appreciated that by the term “added”, as used herein it is meant any addition(s) of amino acid residues to the sequences described herein. For example, the variant antibodies of the invention may be extended at their N-terminus and/or C-terminus with various identical or different amino acid residues.

Variants also encompass various amino acid substitutions. An amino acid “substitution” is the result of replacing one amino acid with another amino acid which has similar or different structural and/or chemical properties. Amino acid substitutions may be made on the basis of similarity in polarity, charge, solubility, hydrophobicity, hydrophilicity, and/or the amphipathic nature of the residues involved.

Variants further encompass conservative amino acid substitutions. Conservative substitution tables providing functionally similar amino acids are well known in the art. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid.

Each of the following eight groups contains other exemplary amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M).

Conservative nucleic acid substitutions are nucleic acid substitutions resulting in conservative amino acid substitutions as defined above.

As used herein, the term “amino acid” or “amino acid residue” refers to naturally occurring and synthetic amino acids, as well as amino acid analogs and amino acid mimetics that function in a manner similar to the naturally occurring amino acids.

Variant sequences refer to amino acid or nucleic acids sequences that may be characterized by the percentage of the identity of their amino acid or nucleotide sequences, respectively, with the amino acid or nucleotide sequences described herein, while maintaining the biological activity (namely the amino acid or nucleotide sequences of the heavy and light chains of the antibodies herein described).

Therefore in some embodiment variant sequences as herein defined refer to nucleic acid sequences that encode the heavy and light chain variable regions, each having a sequence of nucleotides with at least 70% or 75% of sequence identity, around 80% or 85% of sequence identity, around 90%, 91%, 92%, 93%, 94%, 95%, 96%, 97%, 98% or 99% of sequence identity when compared to the sequences of the heavy and light chain variable regions described herein.

In some embodiments the isolated monoclonal antibody or any antigen-binding fragment thereof according to the invention is wherein said antibody comprises a heavy chain variable region and a light chain variable region, wherein said heavy chain variable region is encoded by a nucleic acid sequence which is at least 70% identical to the nucleic acid sequence denoted by SEQ ID NO. 156 or SEQ ID NO. 166 and wherein said light chain variable region is encoded by a nucleic acid sequence which is at least 70% identical to SEQ ID NO. 161 or SEQ ID NO. 171.

In other embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 156 and wherein the light chain variable region of the antibody is encoded by a nucleic acid sequence which is at least 70% identical to SEQ ID NO. 161.

In other embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 166 and wherein the light chain variable region of the antibody is encoded by a nucleic acid sequence which is at least 70% identical to SEQ ID NO. 171. In other embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 106 and wherein the light chain variable region of the antibody is encoded by a nucleic acid sequence which is at least 70% identical to SEQ ID NO. 111.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 76 and wherein the light chain variable region of the antibody is encoded by a nucleic acid sequence which is at least 70% identical to SEQ ID NO. 81.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 57 and wherein the light chain variable region of the antibody is encoded by a nucleic acid sequence which is at least 70% identical to SEQ ID NO. 62.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 67 and wherein the light chain variable region of the antibody is encoded by a nucleic acid sequence which is at least 70% identical to SEQ ID NO. 71.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 86 and wherein the light chain variable region of the antibody is encoded by a nucleic acid sequence which is at least 70% identical to SEQ ID NO. 91.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 96 and wherein the light chain variable region of the antibody is encoded by a nucleic acid sequence which is at least 70% identical to SEQ ID NO. 101.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 116 and wherein the light chain variable region of the antibody is encoded by a nucleic acid sequence which is at least 70% identical to SEQ ID NO. 121.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 126 and wherein the light chain variable region of the antibody is encoded by a nucleic acid sequence which is at least 70% identical to SEQ ID NO. 131.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 136 and wherein the light chain variable region of the antibody is encoded by a nucleic acid sequence which is at least 70% identical to SEQ ID NO. 141.

In further embodiments the isolated monoclonal antibody or any antigen-binding fragment thereof according to the invention is wherein the heavy chain variable region of the antibody is encoded by a nucleic acid sequence which is at least 70% identical to the nucleic acid sequence denoted by SEQ ID NO. 146 and wherein the light chain variable region of the antibody is encoded by a nucleic acid sequence which is at least 70% identical to SEQ ID NO. 151.

In further embodiments the isolated monoclonal antibody according to the invention is wherein said antibody comprises a heavy chain variable region comprising the amino acid sequence denoted by SEQ ID NO. 157, SEQ ID NO. 167 or a variant thereof and a light chain variable region comprising the amino acid sequence denoted by SEQ ID NO. 162, SEQ ID NO. 172 or a variant thereof.

In specific embodiments the isolated monoclonal antibody according to the invention comprises a heavy chain variable region comprising the amino acid sequence denoted by SEQ ID NO. 157 or a variant thereof and a light chain variable region comprising the amino acid sequence denoted by SEQ ID NO. 162, or a variant thereof.

In other embodiments the isolated monoclonal antibody according to the invention comprises a heavy chain variable region comprising the amino acid sequence denoted by SEQ ID NO. 167 or a variant thereof and a light chain variable region comprising the amino acid sequence denoted by SEQ ID NO. 172, or a variant thereof.

In other embodiments the isolated monoclonal antibody according to the invention comprises a heavy chain variable region comprising the amino acid sequence denoted by SEQ ID NO. 107 or a variant thereof and a light chain variable region comprising the amino acid sequence denoted by SEQ ID NO. 112, or a variant thereof.

In other embodiments the isolated monoclonal antibody according to the invention comprises a heavy chain variable region comprising the amino acid sequence denoted by SEQ ID NO. 77 or a variant thereof and a light chain variable region comprising the amino acid sequence denoted by SEQ ID NO. 82, or a variant thereof.

In other embodiments the isolated monoclonal antibody according to the invention comprises a heavy chain variable region comprising the amino acid sequence denoted by SEQ ID NO. 58 or a variant thereof and a light chain variable region comprising the amino acid sequence denoted by SEQ ID NO. 63, or a variant thereof.

In other embodiments the isolated monoclonal antibody according to the invention comprises a heavy chain variable region comprising the amino acid sequence denoted by SEQ ID NO. 68 or a variant thereof and a light chain variable region comprising the amino acid sequence denoted by SEQ ID NO. 72, or a variant thereof.

In other embodiments the isolated monoclonal antibody according to the invention comprises a heavy chain variable region comprising the amino acid sequence denoted by SEQ ID NO. 87 or a variant thereof and a light chain variable region comprising the amino acid sequence denoted by SEQ ID NO. 92, or a variant thereof.

In other embodiments the isolated monoclonal antibody according to the invention comprises a heavy chain variable region comprising the amino acid sequence denoted by SEQ ID NO. 97 or a variant thereof and a light chain variable region comprising the amino acid sequence denoted by SEQ ID NO. 102, or a variant thereof.

In other embodiments the isolated monoclonal antibody according to the invention comprises a heavy chain variable region comprising the amino acid sequence denoted by SEQ ID NO. 117 or a variant thereof and a light chain variable region comprising the amino acid sequence denoted by SEQ ID NO. 122, or a variant thereof.

In other embodiments the isolated monoclonal antibody according to the invention comprises a heavy chain variable region comprising the amino acid sequence denoted by SEQ ID NO. 127 or a variant thereof and a light chain variable region comprising the amino acid sequence denoted by SEQ ID NO. 132, or a variant thereof.

In other embodiments the isolated monoclonal antibody according to the invention comprises a heavy chain variable region comprising the amino acid sequence denoted by SEQ ID NO. 137 or a variant thereof and a light chain variable region comprising the amino acid sequence denoted by SEQ ID NO. 142, or a variant thereof.

In other embodiments the isolated monoclonal antibody according to the invention comprises a heavy chain variable region comprising the amino acid sequence denoted by SEQ ID NO. 147 or a variant thereof and a light chain variable region comprising the amino acid sequence denoted by SEQ ID NO. 152, or a variant thereof.

As demonstrated in FIG. 14, all of the monoclonal antibodies prepared as described herein showed high binding affinity to HCV E2 and a high neutralizing activity.

As described in the appended Examples, human antibodies were raised against HCV E2.

The term “human antibody” as used herein refers to an antibody that possesses an amino acid sequence corresponding to that of an antibody produced by a human and/or has been made using any of the techniques for making human antibodies disclosed herein.

Preparation of human antibodies is well known in the art, for example as described below.

The present invention further encompasses any antigen-binding fragments of the isolated monoclonal antibody of the invention. Such antigen-binding fragments may be for example Fab and F(ab′)2, which are capable of binding antigen. Such fragments may be produced by any method known in the art, for example by proteolytic cleavage, using enzymes such as papain (to produce Fab fragments) or pepsin (to produce F(ab′)2 fragments).

Therefore in some embodiments the isolated monoclonal antibody according to the invention is wherein said antibody is an antibody fragment selected from the group consisting of Fv, single chain Fv (scFv), heavy chain variable region, light chain variable region, Fab, F(ab)₂′ and any combination thereof.

In one embodiment, for constructing a full-length antibody, the variable light chain region and the variable heavy chain regions of the antibody are recovered from scFv molecules. In another embodiment, the variable light chain region is recovered from an scFv molecule and the variable heavy chain region is custom-synthesized based on bioinformatics analysis.

As exemplified below, the human antibodies prepared in accordance with the present disclosure were shown to neutralize HCV using an in vitro FFU (focus-forming unit) neutralization assay in Huh7.5 cells using HCVcc HJ3-5 chimeric virus or viruses containing E2 from genotypes 1-7 (1a (H77/JFH1); 2b (J8/JFH1); 3a (S52/JFH1); 4a (ED43/JFH1); 5a (SA13/JFH1); 6a (HK6a/JFH1); 7a (QC69/JFH1)). The percent neutralization was calculated as the percent reduction in FFU compared with virus incubated with an irrelevant control antibody.

Thus in some embodiments the isolated monoclonal antibody according to the invention is wherein said antibody is a neutralizing antibody.

The term “Neutralizing antibody” (or Nab) as herein defined refers to an antibody which defends a cell from an antigen or infectious body by inhibiting or neutralizing the biological effect of the antigen or infectious body. Neutralizing antibodies are mainly defined by their in vitro activity, which in the present case may be assessed for example by the FFU neutralization assay.

In yet another one of its aspects the present invention provides an isolated nucleic acid molecule comprising a nucleotide sequence encoding an antibody or any antigen-binding fragment thereof as herein defined.

The term “nucleic acid” or “nucleic acid molecule” as herein defined refers to a polymer of nucleotides, which may be either single- or double-stranded, which is a polynucleotide such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides. The term DNA used herein also encompasses cDNA, i.e. complementary or copy DNA produced from an RNA template by the action of reverse transcriptase (RNA-dependent DNA polymerase).

In still another one of its aspects the present invention provides an expression vector comprising the isolated nucleic acid molecule as herein defined.

The term “Expression vector” sometimes referred to as “expression vehicle” or “expression construct”, as used herein, encompass vectors such as plasmids, viruses, bacteriophage, integratable DNA fragments, and other vehicles, which enable the integration of DNA fragments into the genome of the host. Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. The expression vector in accordance with the invention may be competent with expression in bacterial, yeast, or mammalian host cells, to name but few. Non limiting examples include the pMAZ-IgH, pMAZ-IgL and pCC16 vectors, as described below.

The present invention further provides a host cell transfected with the isolated nucleic acid molecule or with the expression vector as herein defined.

The term “host cells” as used herein refers to cells which are susceptible to the introduction of the isolated nucleic acid molecule according to the invention or with the expression vectors according to the invention. Preferably, said cells are mammalian cells, for example 293T cells (which were used in the present disclosure). Transfection of the isolated nucleic acid molecule or the expression vector according to the invention to the host cell may be performed by any method known in the art.

In another one of its aspects the present invention provides a bispecific molecule comprising the antibody as herein defined.

By the term “bispecific molecule” as herein defined it is meant a molecule comprising a first entity being an antibody or any antigen binding fragment thereof as herein defined and a second entity. The second entity may be a second antibody or antigen binding fragment thereof that specifically binds to a different target, such as but not limited to an epitope in HCV-E2 that is different from the epitope recognized by the antibodies in accordance with the invention. The second antibody or antigen binding fragment thereof may also target other HCV proteins, or a host antigen (e.g. a molecule associated with a cell of the immune system). Bispecific antibodies include cross-linked or “heteroconjugate” antibodies and can be made using any convenient cross-linking or recombinant methods.

In yet another one of its aspects the present invention provides an immunoconjugate comprising the antibody or any antigen-binding fragment thereof as herein defined and an additional anti-HCV agent.

The term “immunoconjugate” as herein defined refers to an antibody or any antigen-binding fragment thereof according to the invention that is conjugated (linked or joined) to an additional agent Immunoconjugates may be prepared by any method known to a person skilled in the art, for example, by cross-linking the additional agent to the antibody according to the invention or by recombinant DNA methods.

The term “additional anti-HCV agent” as herein defined refers to any agent known in the art for the treatment of HCV, including but not limited to an additional antibody.

The term “additional antibody” as herein defined refers to an antibody, which is not the antibody according to the invention, which may be used in combination with any one of the antibodies of the invention. Such antibody may be directed against HCV E2, against a different antigen of HCV, or against a host-related moiety.

The present invention further provides a pharmaceutical composition comprising as an active ingredient the isolated monoclonal antibody or any antigen-binding fragment thereof as herein defined, the bispecific molecule or the immunoconjugate according to the invention and a pharmaceutically acceptable carrier, excipient or diluent.

The “pharmaceutical composition” of the invention generally comprises the antibody or any antigen-binding fragment thereof as herein defined and a buffering agent, an agent which adjusts the osmolarity of the composition and optionally, one or more pharmaceutically acceptable carriers, excipients and/or diluents as known in the art. Supplementary active ingredients can also be incorporated into the compositions, e.g. other anti HCV drugs or agents.

As used herein the term “pharmaceutically acceptable carrier, excipient or diluent” includes any and all solvents, dispersion media, coatings, antibacterial and antifungal agents and the like, as known in the art. The carrier can be solvent or dispersion medium containing, for example, water, ethanol, polyol (for example, glycerol, propylene glycol, and liquid polyethylene glycol, and the like), suitable mixtures thereof, and vegetable oils. Each carrier should be both pharmaceutically and physiologically acceptable in the sense of being compatible with the other ingredients and not injurious to the subject. Except as any conventional media or agent is incompatible with the active ingredient, its use in the therapeutic composition is contemplated.

In some embodiments the pharmaceutical composition as herein defined further comprises an adjuvant.

An “adjuvant” as herein defined refers to a pharmacological and/or immunological agent that modifies the effect of other agents. Adjuvants are inorganic or organic chemicals, macromolecules or entire cells of certain killed bacteria, which enhance the immune response to an antigen. Examples of adjuvants include, but are not limited to Freund's adjuvant, aluminum hydroxide etc.

In some further embodiments the pharmaceutical composition according to the invention further comprises an additional anti-HCV agent.

In yet another one of its aspects, the present invention provides the isolated monoclonal antibody or any antigen-binding fragment thereof according to the invention, the bispecific molecule, the immunoconjugate or the pharmaceutical composition as herein defined for use in a method of prophylaxis, treatment or amelioration of HCV infection.

In some further embodiments, the isolated monoclonal antibody or any antigen-binding fragment thereof according to the invention, the bispecific molecule, the immunoconjugate or the pharmaceutical composition as herein defined is for use in a method of prophylaxis, treatment or amelioration of diseases associated with HCV infection.

In some specific embodiments, such diseases may be hepatitis, fibrosis, cirrhosis, liver failure, hepatocellular carcinoma (HCC) or HCV infection post liver transplantation.

Further provided is a method of prophylaxis, treatment, passive immunization or amelioration of HCV infection or diseases associated with HCV infection as described above, comprising administering to a subject in need thereof a therapeutically effective amount of the isolated monoclonal antibody or any antigen-binding fragment thereof according to the invention, the bispecific molecule, the immunoconjugate or the pharmaceutical composition as herein defined.

By the term “prophylaxis” as herein defined it is meant to provide a preventive or prophylactic treatment, namely acting in a protective manner, to defend against or prevent HCV infection, namely before exposure to HCV. Non-limiting examples include post-exposure prophylaxis for accidental needle-stick or other percutaneous or mucosal exposure, prevention of recurrent HCV infection in a transplanted liver.

The terms “treatment”, “treating”, “treat”, “passive immunization”, or forms thereof as used herein, mean preventing, ameliorating or delaying the onset of one or more clinical indications of infection or disease activity resulting from HCV infection in a subject.

Administration according to the present invention may be performed by any of the following routes: oral administration, intravenous, intramuscular, intraperitoneal, intratechal or subcutaneous injection; intrarectal administration; intranasal administration, ocular administration or topical administration.

In specific embodiments administration according to the present invention may be performed intravenously.

In some embodiments the method according to the invention further comprises administering to a subject in need thereof an additional anti-HCV agent. Non limiting examples include pegylated interferon-α, ribavirin, and an HCV protease inhibitor, e.g. boceprevir or telaprevir. In one embodiment, the method of the invention comprises treatment or passive immunization with combinations of anti HCV monoclonal antibodies possessing well-defined and differing epitope specificities in order to overcome virus resistance.

The term “subject in need thereof” as herein defined means primates and particularly humans at risk of being exposed to HCV (e.g. patients receiving a liver transplant) or that have been infected with HCV.

The method according to the invention may be applied where the isolated monoclonal antibody or any antigen-binding fragment thereof, bispecific molecule, immunoconjugate or pharmaceutical composition as herein defined is administered to the subject prior to or after HCV infection.

The term “therapeutically effective amount” for purposes herein defined is determined by such considerations as are known in the art in order to cure, arrest or at least alleviate or ameliorate the medical conditions associated with HCV infection. For any preparation used in the methods of the invention, the dosage or the therapeutically effective amount can be estimated initially from in vitro cell culture assays or known clinical correlates.

In the above and other embodiments the isolated monoclonal antibody or any antigen-binding fragment thereof is administered at a therapeutically effective amount of 10-1000 μg/kg.

It should be appreciated that the therapeutically effective amount as herein defined refers to the isolated monoclonal antibody or any antigen-binding fragment per se, as the active ingredient of a pharmaceutical composition, or as a component of a bispecific molecule or an immunoconjugate.

In some further embodiments the isolated monoclonal antibody or any antigen-binding fragment thereof, bispecific molecule, immunoconjugate or pharmaceutical composition as herein defined is administered to the subject as a single dose or as multiple doses.

As demonstrated in the appended examples, the antibodies described herein, in particular the antibodies referred to as VH510520 (RMS28) and VH16618 (RMS11) were shown to neutralize HCV in an in vitro FFU (focus-forming unit) neutralization assay.

Thus in still another one of its aspects the present invention provides a method of neutralizing HCV infection comprising administering to a subject in need thereof a therapeutically effective amount of the isolated monoclonal antibody or any antigen-binding fragment thereof, the bispecific molecule, the immunoconjugate or the pharmaceutical composition as herein defined.

By the term “neutralizing” it is meant blocking, preventing or at least reducing the ability of HCV to infect the host cells, in particular human hepatic cells.

The ability of the antibody to neutralize the infectivity of HCV may be monitored by any method known in the art, in particular, using the neutralization assay described herein below. The present invention further provides a method of detecting HCV in a biological sample obtained from a subject, said method comprising:

(a) contacting said biological sample with the isolated monoclonal antibody or any antigen-binding fragment thereof according to the invention, wherein said monoclonal antibody is labeled with a detectable marker; and
(b) detecting said isolated monoclonal antibody or any antigen-binding fragment thereof; wherein the presence of said isolated monoclonal antibody or any antigen-binding fragment thereof indicates the presence of HCV in said biological sample.

Detecting the isolated monoclonal antibody may be performed by any method known to a person skilled in the art based on the detectable marker present on the antibody.

The term “detectable marker” refers to any atom, molecule or a portion thereof, the presence, absence or level of which can be directly or indirectly monitored. Labeling of the antibodies as herein defined may be performed by any method known in the art.

The term “biological sample” as herein defined encompasses fluids, solids and tissues obtained from the subject. The term biological sample also refers to forensic samples.

In another one of its aspects the present invention provides a kit for detecting HCV comprising:
(c) at least one labeled isolated monoclonal antibody or any antigen-binding fragment thereof according to the invention;
(d) means for detection of said labeled isolated monoclonal antibody; and optionally
(e) instructions for use of said kit.

It is appreciated that the term “purified” or “isolated” refers to molecules, such as amino acid or nucleic acid sequences, peptides, polypeptides or antibodies that are removed from their natural environment, isolated or separated. An “isolated antibody” is therefore a purified antibody. As used herein, the term “purified” or “to purify” also refers to the removal of contaminants from a sample.

In a further aspect, the present invention provides a method of preparing neutralizing anti HCV scFv antibodies associated with HCV clearance, comprising the steps of:

a. constructing a phage display scFv antibody library from peripheral blood mononuclear cells (PBMC) obtained from SC individuals;

b. identifying at least one HCV E2 binder;

c. comparing the sequence of the at least one HCV E2 binder with the sequences in stratifying clusters from a general repertoire that are associated with HCV clearance;

d. selecting an scFv sequence with high similarity to a sequence in a stratifying cluster from said general repertoire that is associated with HCV clearance.

As used herein, the term “phage display” relates to a bacteriophage-based laboratory technique. In this technique, a gene encoding a protein of interest is inserted into a phage coat protein gene, causing the phage to “display” the protein on its outside surface while containing the gene for the protein on its inside, resulting in a connection between genotype and phenotype. These displaying phages can then be screened against other proteins, peptides or DNA sequences, in order to detect interaction between the displayed protein and those other molecules. In this way, large libraries of proteins can be screened and amplified in a process called in vitro selection, which is analogous to natural selection. Non-limiting examples of suitable bacteriophages that may be used in phage display are M13, filamentous phages, T4, T7, and λ phages.

The term “HCV E2 binder” as used herein refers to a phage obtained from the library defined above, displaying an antibody that is able to bind to HCV envelope protein E2. Methods for assessing binding are well known in the art, for example ELISA binding assay as shown in Example 5 (specifically see FIG. 12A).

The term “phagemid” as used herein refers to a DNA-based cloning vector, which has both bacteriophage and plasmid properties (see examples in Material and methods). These vectors carry, in addition to the origin of plasmid replication, an origin of replication derived from bacteriophage. Unlike commonly used plasmids, phagemid vectors differ by having the ability to be packaged into the capsid of a bacteriophage, since they have a genetic sequence that signals for packaging.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

### EXAMPLES

Without further elaboration, it is believed that one skilled in the art can, using the preceding description, utilize the present disclosure to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook & Russell, 2001.

Standard medicinal chemistry methods known in the art not specifically described herein are generally followed essentially in the series “Comprehensive Medicinal Chemistry” by various authors and editors, published by Pergamon Press.

Materials and Methods

Cell Lines

Huh-7.5 cells and Huh7/FT3-7 cells are human hepatoma cell lines that are highly permissive for infection and replication of cell culture infectious HCV (HCVcc) (Yi, M., et al. (2007) Journal of virology 81, 629-638). Cells were cultured in Dulbecco's modified Eagle's medium (DMEM) containing high glucose; 10% fetal bovine serum (FBS); 1% L-glutamine; 1% penicillin streptomycin; and 1% non-essential amino acid. The cells were incubated in a humidified incubator at 37° C. containing 5% CO2. The irradiated 3T3-msCD40L feeder cells that express CD40L were obtained from the National Institutes of Health (NIH) and cultured as previously described (Huang, J., et al. (2013) Nature protocols 8, 1907-1915).

Virus

Virus stocks from HJ3-5 chimeric virus (Yi, M., et al. (2007) Journal of virology 81, 629-638) and the other chimeric viruses containing E2 envelope protein from genotypes 1-7: HJ3-5/1a, H77C/1a, j6/2b, s52/3a, ED43/4a, sa13/5a, HK6A/6a, QC69/7a (Gottwein, J. M., et al. (2009) Hepatology (Baltimore, Md. 49, 364-377), were produced in Huh7/FT3-7 cells and viral titers were determined by FFU assay in Huh-7.5 cells, as described previously (Yi, M., et al. (2007) Journal of virology 81, 629-638).

Antibodies

A panel of HCV mnAbs CBH-4B, CBH-4D, HC-1, HC-11, CBH-7, HC84.22, HC84.26, HC33.1, and HC33.4 that are representative E2 antigenic domain A-E antibodies, and a control nonspecific antibody R04 (Pierce, B. G., et al. (2016) PNAS) and reviewed previously (Kong, L., et al. (2015) Current opinion in virology 11, 148-157; Ball, J. K., et al. (2014) Antiviral research 105, 100-111).

Sample Collection

All blood samples were collected from the Liver Institute at Belinson and the Galilee Medical Center, Israel. In total, we obtained blood samples from 80 individuals; of these, 18 were individuals that spontaneously cleared HCV infection, 52 were with persistent chronic HCV infections, and 10 were from healthy controls. Subjects were defined as spontaneously cleared HCV if anti-HCV antibodies are detectable, with undetectable HCV RNA assessed by the Taqman reverse-transcription polymerase chain reaction (RT-PCR) quantitative assays. HCV chronic infections were defined as viremia if there were detectable viral loads for more than 1 year. Both cohorts were not treated with any anti-viral treatment. All blood samples were collected using protocols approved by the Institutional Review Boards and were in accordance with the ethical standards of the Helsinki Declaration. Sample data are summarized in Table 1. For the isolation of peripheral blood mononuclear cells (PBMCs), 30-50 ml of whole blood from each donor was separated on Ficoll-Paque gradient (Lymphoprep™) according to the manufacturer's instructions.

TABLE 1
Summary of sample data

| | | | | Analysis |
|---|---|---|---|---|
| Chronic HCV (CI)/ | | | | (General repertoire/ |
| Cleared HCV (SC)/ | Sample | | HCV | specific repertoire/ |
| Control (C) | ID | Sex | genotype | Library) |
| Chronic HCV (CI) | CI1 | Female | 1b | Library |
| | CI2 | Female | 1b | Library |
| | CI3 | Female | 1b | Library |
| | CI4 | Female | 1b | Library, General |
| | | | | repertoire |
| | CI5 | Female | 1b | Library |
| | CI6 | Female | 1b | Library, General |
| | | | | repertoire |
| | CI7 | Female | 1b | Library, General |
| | | | | repertoire |
| | CI8 | Male | 1b | Library |
| | CI9 | Male | 6g | Library |
| | CI10 | Female | 3a | Library, General |
| | | | | repertoire |
| | CI11 | Male | 1b | Library, General |
| | | | | repertoire |
| | CI12 | Female | 1b | Library |
| | CI13 | Male | 3a | Library, General |
| | | | | repertoire |
| | CI14 | Male | 3a | Library |
| | CI15 | Male | 1b | Library, General |
| | | | | repertoire |
| | CI16 | Male | 1a | Library, General |
| | | | | repertoire |
| | CI17 | Female | 1b | Library, General |
| | | | | repertoire |
| | CI18 | male | 1b | Library |
| | CI19 | male | 1b | Library |
| | CI20 | Female | 3a | Library, General |
| | | | | repertoire |
| | CI21 | Male | 1b | Library, General |
| | | | | repertoire |
| | CI22 | Male | 1b | Library, General |
| | | | | repertoire |
| | CI23 | Male | 1b | Library, General |
| | | | | repertoire |
| | CI25 | Female | 1b | General repertoire |
| | CI26 | Male | 1b | General repertoire |
| | CI51 | Female | 1b | Specific repertoire |
| | CI55 | Male | 1a | Specific repertoire |
| | CI56 | Male | 1b | Specific repertoire |
| | CI57 | Female | 1b | Specific repertoire |
| | CI58 | Male | 1b | Specific repertoire |
| | CI59 | Female | 1b | Specific repertoire |
| | CI60 | Male | 1b | Specific repertoire |
| | CI61 | Female | 1b | Specific repertoire |
| | CI65 | Male | 1 | Specific repertoire |
| | CI66 | Male | 1 | Specific repertoire |
| Cleared HCV (SC) | SC1 | Female | N/A | Library, General |
| | | | | repertoire |
| | SC2 | Female | N/A | Library, General |
| | | | | repertoire |
| | SC3 | Male | N/A | Library, General |
| | | | | repertoire |
| | SC4 | Male | N/A | Library |
| | SC5 | Female | N/A | Library |
| | SC6 | Male | N/A | Library |
| | SC7 | Male | N/A | Library, General |
| | | | | repertoire |
| | SC8 | Male | N/A | Library, General |
| | | | | repertoire |
| | SC9 | Female | N/A | General repertoire |
| | SC10 | Male | N/A | General repertoire |
| | SC11 | Female | N/A | General repertoire |
| | SC12 | Female | N/A | General repertoire |
| | SC14 | Female | N/A | Library, General |
| | | | | repertoire |
| | SC15 | Female | N/A | General repertoire, |
| | | | | Specific repertoire |
| | SC16 | Female | N/A | Specific repertoire |
| | SC17 | Female | N/A | Specific repertoire |
| | SC18 | Female | N/A | Specific repertoire |
| Control (C) | C1 | Male | N/A | Specific repertoire |
| | C2 | Male | N/A | Specific repertoire |
| | C3 | Male | N/A | Specific repertoire |
| | C4 | Female | N/A | General repertoire |
| | C5 | Female | N/A | General repertoire |
| | C6 | Male | N/A | General repertoire |
| | C7 | Male | N/A | General repertoire |
| | C8 | Female | N/A | General repertoire |
| | C9 | Male | N/A | General repertoire |
| | C10 | Male | N/A | General repertoire |

Expression and Purification of the E2 Glycoprotein

The H77 genotype 1a E2 sequence (GenBank accession no. AF009606), spanning residues 384-661 (not containing the transmembrane domain), was amplified by PCR using HCV plasmid pHJ3-5 (Yi, M., et al. (2007) Journal of virology 81, 629-638) and primers pSHOOTER-sec-E2-1a-SE and pSHOOTER-sec-E2-1a-As (primers are listed in Table 2. The PCR product was digested with Notl and Ncol and cloned into plasmid pCMV-SEC-MBP containing signal peptide for secretion, His and Myc tags, and fused to maltose-binding protein (MBP) for higher expression and stabilization. The resulting plasmid was termed pCMV-SEC-MBP-E2-384-661-1a-His-Myc.

For production of E2 protein, 293T cells were transfected with 12 μg pCMV-SEC-MBP-E2-384-661-1a-His-Myc expression plasmid by PEI transfection reagent. At 72 h post transfection, medium containing the secreted protein was collected from cells for protein purification. The E2 protein was purified using Ni-NTA agarose beads (Qiagen) according to the manufacturer's instructions. Purified E2 glycoprotein was stored at −20° C. E2 glycoprotein-containing fractions were analyzed on SDS 10% polyacrylamide gels.

TABLE 2
List of primers

| Primer's name | Sequence | SEQ ID NO: |
|---|---|---|
| pSHOOTER-sec-E2-1a-SE | GGGAAAGGTACCGTCCTCTCTCGTGATCGAGGGTAGGC | 1 |
| | CTGAATTCAGTACCATGGCCGAAACCCACGTCACCGG | |
| pSHOOTER-sec-E2-1a-AS | GGGAATGCGGCCGCCTCGGACCTGTCCCTG | 2 |
| TAB-RI | CCATGATTACGCCAAGCTTGGGAGCC | 3 |
| CBD-As | GAATTCAACCTTCAAATTGCC | 4 |
| Hu-VH-NcoI-BACK1-1 | TTTAAGCCATGGCCCAGGTBCAGCTKGTRCARTCTGG | 5 |
| Hu-VH-NcoI-BACK1-2 | TTTAAGCCATGGCCCARATGCAGCTGGTGCAGTCTGG | 6 |
| Hu-VH-NcoI-BACK1-3 | TTTAAGCCATGGCCGARGTSCAGCTGGTRCAGTCTGG | 7 |
| Hu-VH-NcoI-BACK2-1 | TTTAAGCCATGGCCCAGATCACCTTGAAGGAGTCTGG | 8 |
| Hu-VH-NcoI-BACK2-2 | TTTAAGCCATGGCCCAGGTCACCTTGAGGGAGTCTGG | 9 |
| Hu-VH-NcoI-BACK2-3 | TTTAAGCCATGGCCCAGGTCACCTTGAAGGAGTCTGG | 10 |
| Hu-VH-NcoI-BACK3-1 | TTTAAGCCATGGCCGARGTRCARCTGGTGGAGTCYGG | 11 |
| Hu-VH-NcoI-BACK3-2 | TTTAAGCCATGGCCCAGGTGCAGCTGGTGGAGTCTGG | 12 |
| Hu-VH-NcoI-BACK3-3 | TTTAAGCCATGGCCGAGGTGCAGCTGTTGGAGTCTGG | 13 |
| Hu-VH-NcoI-BACK3-4 | TTTAAGCCATGGCCGAGGTGCAGCTGGTGGAGWCTG | 14 |
| Hu-VH-NcoI-BACK4-1 | TTTAAGCCATGGCCCAGGTGCARCTGCAGGAGTCGGG | 15 |
| Hu-VH-NcoI-BACK4-2 | TTTAAGCCATGGCCCAGCTGCAGCTGCAGGAGTCSGG | 16 |
| Hu-VH-NcoI-BACK6-1 | TTTAAGCCATGGCCCAGGTACAGCTGCAGCAGTCAGG | 17 |
| Hu-JH-FORF-2-4-5 | TCCTGCTGAGCCTGAGGAGACRGTGACCAGGGTKCC | 18 |
| Hu-JH-FORF3 | TCCTGCTGAGCCTGAAGAGACGGTGACCATTGTCCC | 19 |
| Hu-JH-FORF6 | TCCTGCTGAGCCTGAGGAGACGGTGACCGTGGTCCC | 20 |
| Hu-JH-FORF-2-4-5L | CCACCACCACCGGATCCTCCTCCTCCTGCTGAGCCTGA | 21 |
| | GGAGACRGTGACCAGGGTKCC | |
| Hu-JH-FORF3L | CCACCACCACCGGATCCTCCTCCTCCTGCTGAGCCTGA | 22 |
| | AGAGACGGTGACCATTGTCCC | |
| Hu-JH-FORF6L | CCACCACCACCGGATCCTCCTCCTCCTGCTGAGCCTGA | 23 |
| | GGAGACGGTGACCGTGGTCCC | |
| Hu-VK-BACKF1S | GGCGGCGGCTCCRHCATCYRGWTGACCCAGTC | 24 |
| Hu-VK-BACKF2-4S | GGCGGCGGCTCCGAYRTYGTGATGACYCAGWC | 25 |
| Hu-VK-BACKF3S | GGCGGCGGCTCCGAAATWGTRWTGACRCAGTC | 26 |
| Hu-VK-BACKF5S | GGCGGCGGCTCCGAAACGACACTCACGCAGTC | 27 |
| Hu-VK-BACKF6S | GGCGGCGGCTCCGAWRTTGTGMTGACWCAGTC | 28 |
| Hu-VK-Lin-BACKF1L | GGATCCGGTGGTGGTGGTTCCGGAGGCGGCGGCTCCGG | 29 |
| | CGGCGGCTCCRHCATCYRGWTGACCCAGTC | |
| Hu-VK-Lin-BACKF2-4L | GGATCCGGTGGTGGTGGTTCCGGAGGCGGCGGCTCCGG | 30 |
| | CGGCGGCTCCGAYRTYGTGATGACYCAGWC | |
| Hu-VK-Lin-BACKF3L | GGATCCGGTGGTGGTGGTTCCGGAGGCGGCGGCTCCGG | 31 |
| | CGGCGGCTCCGAAATWGTRWTGACRCAGTC | |
| Hu-VK-Lin-BACKF5L | GGATCCGGTGGTGGTGGTTCCGGAGGCGGCGGCTCCGG | 32 |
| | CGGCGGCTCCGAAACGACACTCACGCAGTC | |
| Hu-VK-L-BACKF6L | GGATCCGGTGGTGGTGGTTCCGGAGGCGGCGGCTCCGG | 33 |
| | CGGCGGCTCCGAWRTTGTGMTGACWCAGTC | |
| Hu-JK-NotI-FORF1-3-4 | ATATATGCGGCCGCTTTGATHTCCACYTTGGTCC | 34 |
| Hu-JK-NotI-FORF2 | ATATATGCGGCCGCTTTGATCTCCAGCTTGGTCC | 35 |
| Hu-JK-NotI-FORF5 | ATATATGCGGCCGCTTTAATCTCCAGTCGTGTCC | 36 |
| Hu-VL-BACKF1S | GGCGGCGGCTCCCAGTCTGTSBTGACKCAGCC | 37 |
| Hu-VL-BACKF2S | GGCGGCGGCTCCCAGTCTGCCCTGACTCAGCC | 38 |
| Hu-VL-BACKF3S | GGCGGCGGCTCCTCYTMTGWGCTGACWCAGCC | 39 |
| Hu-VL-BACKF3DEGS | GGCGGCGGCTCCTCCTATGAGCTGAYHCAGSWVC | 40 |
| Hu-VL-BACKF4-5 | GGCGGCGGCTCCCAGSYTGTGCTGACTCAAYC | 41 |
| Hu-VL-BACKF6S | GGCGGCGGCTCCAATTTTATGCTGACTCAGCC | 42 |
| Hu-VL-BACKF7-8S | GGCGGCGGCTCCCAGRCTGTGGTGACYCAGG | 43 |
| Hu-VL-BACKF9-10S | GGCGGCGGCTCCCWGSCWGKGCTGACTCAGCC | 44 |
| Hu-VL-BACKF1L | GGATCCGGTGGTGGTGGTTCCGGAGGCGGCGGCTCCGG | 45 |
| | CGGCGGCTCCCAGTCTGTSBTGACKCAGCC | |
| Hu-VL-BACKF2L | GGATCCGGTGGTGGTGGTTCCGGAGGCGGCGGCTCCGG | 46 |
| | CGGCGGCTCCCAGTCTGCCCTGACTCAGCC | |
| Hu-VL-BACKF3L | GGATCCGGTGGTGGTGGTTCCGGAGGCGGCGGCTCCGG | 47 |
| | CGGCGGCTCCTCYTMTGWGCTGACWCAGCC | |
| Hu-VL-BACKF3DEGL | GGATCCGGTGGTGGTGGTTCCGGAGGCGGCGGCTCCGG | 48 |
| | CGGCGGCTCCTCCTATGAGCTGAYHCAGSWVC | |
| Hu-VL-BACKF4-5L | GGATCCGGTGGTGGTGGTTCCGGAGGCGGCGGCTCCGG | 49 |
| | CGGCGGCTCCCAGSYTGTGCTGACTCAAYC | |
| Hu-VL-BACKF6L | GGATCCGGTGGTGGTGGTTCCGGAGGCGGCGGCTCCGG | 50 |
| | CGGCGGCTCCAATTTTATGCTGACTCAGCC | |
| Hu-VL-BACKF7-8L | GGATCCGGTGGTGGTGGTTCCGGAGGCGGCGGCTCCGG | 51 |
| | CGGCGGCTCCCAGRCTGTGGTGACYCAGG | |
| Hu-VL-BACKF9-10L | GGATCCGGTGGTGGTGGTTCCGGAGGCGGCGGCTCCGG | 52 |
| | CGGCGGCTCCCWGSCWGKGCTGACTCAGCC | |
| Hu-JL-NotI-FORF1-2-3 | ATATATGCGGCCGCTAGGACGGTSACCTTSGTCCC | 53 |
| Hu-JL-NotI-FORF4 | ATATATGCGGCCGCTAGGACGATCAGCTGGGTTCC | 54 |
| Hu-JL-NotI-FORF5 | ATATATGCGGCCGCTAGGACGGTCAGCTCSGTCCC | 55 |
| Hu-JL-NotI-FORF6-7 | ATATATGCGGCCGCTAGGACGGTCASCTKGGTKSS | 56 |

Construction of an immune anti-HCV antibody phage display library A phage display antibody library was constructed from a source of pooled PBMCs obtained from 10 SC patients. For library construction, a degenerative primer set was designed by using the IMGT database (IMGT®, the international ImMunoGeneTics information System® http://www.imgt.org) (Lefranc, M. P., et al. (1999) Nucleic acids research 27, 209-212) (primers are listed in Table 2). The phage antibody library was produced using a protocol as previously described (Nahary, L., et al. (2009) Methods in molecular biology 525, 61-80). In brief, total RNA was extracted from 10⁷ PBMCs using the RNeasy mini kit (Qiagen). cDNA was produced from mRNA by reverse transcription using the AccuScript Hi-Fi cDNA Synthesis Kit (Agilent). Heavy and light chain variable domains were amplified from the RT-PCR cDNA product by PCR using the primer sets. The heavy variable domains were amplified using the primer sets Hu-VH1-6-Ncol-BACK and Hu-JH1-6-FORF and the light variable domain was amplified using primer sets Hu-VK1-6-BACKF and Hu-JK1-5-Notl-FORF (for amplifying Kappa light chains) or Hu-VL1-10-BACKF and Hu-JL1-7-Notl-FORF (for amplifying Lambda light chains). For the combinatorial assembly of the heavy and light chain variable domains into complete single-chain variable fragments (scFv), the fragments were mixed according to their natural frequencies, and PCR was performed using the assembly primer (forward) and the primers set Hu-JK1-5-Notl-FORF for Kappa scFv or the primers set Hu-JL1-7-Notl-FORF for Lambda scFv (reverse) (primers are listed in Table 2). The amplified scFvs were cloned into the phagemid vector pCC16 (Nahary, L., et al. (2009) Methods in molecular biology 525, 61-80). The ligated DNA was used for electroporation into electrocompetent XL-1 cells (Agilent Technologies) under the following conditions: 2.5 kV, 200Ω, 25 μF. In total, 75 electroporations were conducted that yielded a total library size of 6×10⁷ individual clones. To test the diversity of the libraries, the scFv genes were amplify from 30 colonies from the library by PCR. The PCR products were digested by BstNI (NEB). The digested samples were separated on 2.5% agarose gel. A diverse running pattern indicates sequence diversity. Rescue of the library using helper phage and preparation of library stocks was performed essentially as described (Nahary, L., et al. (2009) Methods in molecular biology 525, 61-80).

Biopanning and Isolation of Monoclonal Anti-E2 Phages

To enrich E2-specific phages, five cycles of biopanning were performed for the SC library essentially as described (Nahary, L., et al. (2009) Methods in molecular biology 525, 61-80). In brief, phages were first rescued from the library. Then, the first cycle of enrichment was performed by coating the wells with E2 glycoprotein, and then 10¹¹ phages were added to the wells. Non-specific phages were washed by PBST and then specific phages were eluted with 100 mM triethylamine. For neutralization, 1M Tris.Cl pH 7.4 was added. Eluted phages were used for the next cycle of biopanning Phages were pooled from the 4th and 5th biopanning cycles. Next, 96 colonies were picked from each cycle and rescued essentially as described (Nahary, L., et al. (2009) Methods in molecular biology 525, 61-80). Their specificity to E2 was screened by ELISA, as described below.

Expression and Purification of Full-Length Antibodies

To produce full-length IgGs, the heavy and light chains from scFvs were cloned into pMAZ-IgH and pMAZ-IgL vectors that contain the constant regions of IgG1 and a signal peptide for secretion (Mazor, Y., et al. (2007) Journal of immunological methods 321, 41-59). The variable heavy chain region was recovered by PCR from pCC16 vector, which carries the selected scFv using primers TAB-RI and CBD-As (Table 2). Alternatively, the variable Heavy chain region sequences identified and selected by bioinformatic analysis were custom-synthesized (IDT, Israel). The variable Kappa and Lambda chain regions were recovered by PCR from pCC16 vector, which carries the selected scFv using primers TAB-RI and CBD-As (Table 2). PCR products were digested with BssHII and Nhel for heavy chains, BssHII and Bsiwl for the light Kappa chain, and BssHII and Avrll for the light Lambda chain, and cloned into the appropriate vectors.

For antibody production, 293T cells were transfected with pMAZ-IgH expressing the Heavy chain and with pMAZ-IgL expressing the Light chain. At 72 h post transfection, medium was collected from the cells and antibodies were purified using Protein A Sepharose CL-4B beads (GE healthcare) according to the manufacturer's instructions. Purified antibodies were stored at −20° C. Fractions containing Antibodies were analyzed on SDS 15% polyacrylamide gels.

ELISA

For detecting specific antibodies in patients' sera: Each well of the ELISA plate was coated with 0.5 μg of rE2 diluted in 100 μl of coating buffer and the plates were incubated at 4° C. overnight. The plates were washed twice with PBST and blocked with 3% skim milk in PBS for 1 hr at 37° C. Next, the plates were washed twice with PBST and serum (diluted 1:1000) from different patients were added to the wells, followed by 1 hr incubation at RT. The plates were washed three times with PBST and goat a human HRP-conjugated antibody diluted 1:10000 was added to each well, followed by 1 hr incubation at RT. Then, 100 μl of Tetramethylbenzidine (TMB) was added to each well and following incubation of 5-10 min, the reaction was stopped by adding 50 μl 0.5M of H₂SO₄ to each well. The signal was detected at a wavelength of 450 nm by a plate reader.

For detecting binding phages: ELISA was performed as previously described (43). First, 96-well ELISA plates were coated with 5 μg of rE2 or negative control protein (BSA). Plates were incubated overnight, then washed ×3 with PBS, and blocking buffer was added to the plates for 2 hr at 37° C. Next, individual rescued phages were added from the master plate. Plates were incubated at RT 1 h and washed ×3 with PBS. Next, 1:5000 HRP conjugated to a M13 antibody was added. Then, 100 μl of TMB was added and following an incubation of 30 min, the reaction was stopped by adding 50 μl 0.5M of H₂SO₄ to each well. The signal was detected at a wavelength of 410 nm by a plate reader. Specific phages were picked by detection of positive signal for rE2 compared with BSA.

For determining antibodies' specificity. For detecting antibodies binding to rE2, ELISA plates were coated with 5 μg of rE2. The plate was incubated and blocking buffer was added. Then, antibodies were added in concentration of 16 μg/ml and incubated for 1 hr at RT. HRP-conjugated Goat a Human was added at 1:10000 dilution and the plate was incubated for 1 hr at RT. TMB was added and following an incubation of 5-10 min, 50 μl 0.5M of H₂SO₄ was added to each well. The signal was detected at a wavelength of 450 nm by a plate reader.

Focus-Forming Unit (FFU) Reduction Neutralization Assay

Neutralization assays were carried out essentially as described previously (Gal-Tanamy, et al. (2008) Proceedings of the National Academy of Sciences of the United States of America 105, 19450-19455). Huh7.5 cells were seeded on an eight-chamber slide and incubated overnight at 37° C. The next day, 5×10¹¹ of each selected phage or different concentrations of purified IgGs were incubated for 1 hr with 100 FFU of HCVcc HJ3-5 chimeric virus or viruses containing E2 from genotypes 1-7 (1a (H77/JFH1); 2b (J8/JFH1); 3a (S52/JFH1); 4a (ED43/JFH1); 5a (SA13/JFH1); 6a (HK6a/JFH1); 7a (QC69/JFH1)). Next, phages/IgGs and virus mixtures were added to the wells. The slides were incubated for 24 hr. Next, 200 μl of DMEM was added to each well and the slide was incubated for another 24 hr. Then, the slides were washed twice with 200 μl PBS. The PBS was gently removed and 100 μl of Methanol:Acetone 1:1 was added to each well, followed by 10 minutes incubation at RT. Each well was washed twice with 200 μl PBS. Then 7.5% BSA in PBS was added with serum from a chronically infected HCV patient at a dilution of 1:1000, followed by 1 hour of incubation at 37° C. Each well was washed twice with 200 μl PBS. Next, 100 μl of 7.5% BSA in PBS with fluorescently labeled goat anti-human antibody diluted 1:100 was added to each well, followed by 1 hr of incubation at RT. Each well was washed 3 times with 200 μl PBS. Neutralization was measured by immunofluorescence microscopy, followed by manual counting of foci of infected cells. The percent neutralization was calculated as the percent reduction in FFU compared with virus incubated with an irrelevant control antibody.

Isolation of HCV-Specific B Cells

A platform was established for the propagation and isolation of HCV-specific B cells. PBMCs from CI and SC patients were isolated and CD19⁺ B cells were separated by a FACS sorter. B cells were then plated on feeder irradiated 3T3-msCD40L cells that express CD40L, which induces proliferation, Ab class switching, and secretion (Wykes, M. (2003) Immunology and cell biology 81, 328-331). B cells were activated with 5 μg/ml rE2 protein and a combination of IL2 (10000 U/ml) and IL21 (100 μg/ml) (Berglund, L. et al. (2013) Blood 122, 3940-3950). The combination of CD40L feeder cells and the addition of cytokines IL2 and IL21 can successfully stimulate switched memory B cells to produce high concentrations of IgG to the supernatant.

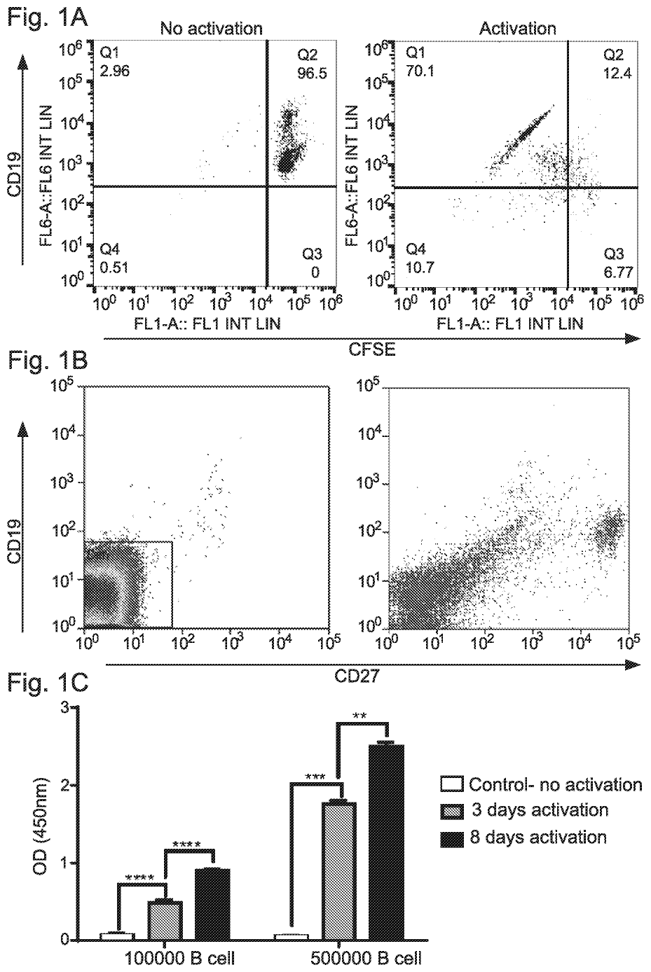

FIG. 1 demonstrates the successful propagation of memory B cells following separation of CD19⁺ B cells from a healthy individual, that were grown on 3T3-msCD40L cells and stimulated with a pool of positive peptides and IL2 and IL-21. Evaluation of CFSE staining following 14 days of culture demonstrates CFSE fading, only under stimulated conditions. This indicates the proliferation of the activated culture (FIG. 1A). Moreover, in the activated culture, 23% of the population was memory B-cells that are positive for CD27+, compared with very low numbers of CD27+ cells in the non-activated culture (FIG. 1B). For evaluating the ability of B cells to differentiate and produce IgG, the concentrations of IgG secreted to the culture medium were measured three or eight days following B-cell activation by ELISA. As shown in FIG. 1C, the activation induced IgG secretion, in a time and cell number-dependent manner.

For isolation of HCV-specific B-cells, B-cells from CI and SC patients were isolated and stimulated as described above. The cultures were incubated for 14 days and then HCV-specific B cells were isolated. Activated B cells were incubated with rE2 and stained with CD19-PE, CD27-BV421, and tagged rE2 (anti-cMyc, alexa fluor 633). Viable CD19+, CD27+, and E2+ were isolated by FACS. These HCV-specific B cells were then grown for one week, as described above. Supernatants were collected at each step and used in the HCV-neutralization assays. The background was compared to healthy individuals, stained and gated as the tested samples.

### Sequencing B-Cell Repertoires

Library Preparation

Total RNA was purified from 5×10⁶ PBMCs from each sample (using RNeasy Midi kit, Qiagen). RT-PCR was performed using an oligo dT primer. An adaptor sequence was added to the 5′ end, which contains a universal priming site and a 17-nucleotide unique molecular identifier (Stern, J. N., et al. (2014) Science translational medicine 6, 248ra107). Products were purified, followed by PCR using primers targeting the IgD, IgM, IgG and IgA regions, and the universal adaptor. PCR products were then purified using AMPure XP beads. A second PCR was performed to add the Illumina P5 adaptor to the constant region end, and a sample-indexed P7 adaptor to the universal adaptor. Final products were purified, quantified with a TapeStation (Agilent Genomics), and pooled in equimolar proportions, followed by 2×300 paired-end sequencing with a 20% PhiX spike on the Illumina MiSeq platform according to the manufacturer's recommendations.

Bioinformatic Analyses

Pre-processing of raw sequencing reads: Repertoire Sequencing TOolkit (pRESTO version 0.5.8) (Vander Heiden, J. A., et al. (2014) Bioinformatics 30, 1930-1932) was applied to the raw reads using the following steps: a. Removal of low-quality reads (mean Phred quality score <20). b. Removal of reads where the primer could not be identified or had a poor alignment score (mismatch rate >0.1). c. Identification of sets of sequences with identical molecular IDs (corresponding to the same mRNA molecule). These are collapsed into one consensus sequence per set, after removing sets with a mean mismatch rate >0.2. d. Assembly of the two consensus paired-end reads into a complete antibody sequence. Then, V(D)J segments were assigned for each of the antibody sequences using IMGT/HighV-QUEST (Alamyar, E., et al. (2012) Methods in molecular biology 882, 569-604). This was followed by quality control and additional filtering: a. Removal of non-functional sequences due to a stop codon or a reading frame shift between the V and the J gene. b. Sequences with CDR3 length <12 nucleotides. c. Samples with an unusually abundant single V-J CDR3 length combination were excluded: samples CI4 and SC12 met this criterion, since they had a single sequence in >50% of the raw reads. d. For mutation analysis sequences with read numbers (CONSCOUNT) lower than two were removed. e. For IGHV gene usage we showed analysis for only functional genes that were in the 15 topmost frequent in at least one sample.

Clustering of Related B-Cell Sequences Across all Samples

Sequences were first grouped according to their V-gene, J-gene, and CDR3 length. For each group, the difference in amino acids between each pair of CDR3s was calculated by Hamming distance. Hierarchical clustering by a complete linkage method was applied and sequences were clustered by genetic distance, using a threshold of 0.15, i.e., the maximal dissimilarity between any two CDR3 sequences in a cluster never exceeded 15%. As an additional quality control step, sequence clusters for which >90% of sequences came from a single sample were removed.

Comparing HCV-Specific B Cells and General Repertoires from SC and CI Clinical Groups by Amino Acid Conservation Levels

The frequency of each amino acid (AA) at each CDR3 position was calculated for each B-cell cluster. The sums of frequency squares were calculated for each clinical group. B-cell clusters containing CDR3 positions for which the sum of frequencies in SC was greater than the corresponding sum for CI by more than 0.5 were selected. Only clusters with sequences originating from more than one sample, and sequences with CONSCOUNT >1 were used.

Prediction Model Based on the Patients' Repertoire

1. Sequences were grouped to clusters as described above.
2. The frequency of each cluster per sample was calculated.
3. A classification model was applied as follows:
a. The data set was randomly divided into 18 (˜90%) and 2 samples (˜10%) of training and test sets, respectively.
b. Feature selection was performed by a random forest model, choosing the most informative 18 features.
c. Logistic regression with an L2 regularization penalty was applied to these 18 remaining features, and the model was applied to the test set. The accuracy rate was measured.
d. The process was repeated 100 times; each time two different samples were taken as a test set.
e. Random predictions: to ensure that our results are not biased, clinical group labels were randomly shuffled. Then, steps a-d were applied to this permuted labels model.
4. A similar model was applied to T-cell repertoires, except that clusters of sequences were defined by identical CDR3 regions at the amino acid level.

The antibody repertoires sequencing datasets for this study were deposited in the European Nucleotide Archive. The accession numbers are ERR2843386-ERR2843427.

The overall approach is summarized in FIG. 2; it included a collection of blood samples from CI and SC HCV infections in addition to healthy controls, and a screen to identify samples containing high levels of HCV-neutralizing antibodies. Selected samples were used for sequencing of total and HCV-specific antibody repertoires, as well as total T-cell receptor repertoires. This was followed by constructing monoclonal antibodies associated with infection clearance, based on phage display antibody library and repertoire data (FIG. 2).

### Example 1: Anti-HCV Antibodies in Resolved Infections are Potent Neutralizers

PBMCs and sera was collected from 80 individuals. Of these, 18 were individuals that spontaneously cleared HCV infection, 52 were with persistent chronic HCV infections, and 10 were from healthy controls.

Subjects were defined as spontaneously cleared HCV if anti-HCV antibodies are detectable, with undetectable HCV RNA assessed by the Taqman reverse-transcription polymerase chain reaction (RT-PCR) quantitative assays. HCV chronic infections were defined as viremia if there were detectable viral loads for more than 1 year. Both cohorts were not treated with any anti-viral treatment. For the isolation of peripheral blood mononuclear cells (PBMCs), 30-50 ml of whole blood from each donor was separated on Ficoll-Paque gradient (Lymphoprep™) according to the manufacturer's instructions.

To validate the presence of neutralizing antibodies in sera from CI and SC HCV infections, these sera were first screened by ELISA for antibodies able to bind a recombinant HCV envelope protein E2 (rE2). Although high levels of anti-rE2 were detected in chronic HCV infections, very low levels were detected in resolved HCV infections (FIG. 3A). This is expected, since the ongoing infection in CI patients results in the generation of large numbers of anti-HCV antibodies from plasma cells, whereas in resolved individuals, anti-HCV antibodies are secreted from lower number of circulating HCV-specific long lived plasma cells or memory B-cells. Then, these sera were screened for HCV-neutralization by performing an HCVcc neutralization assay. Approximately a twofold drop in neutralization efficiency was observed in resolved infections (an average of 45%) compared with chronic infections (an average of 85%) (FIG. 3B).

To validate that indeed HCV-specific immunity was measured, two CI samples were collected before and after successful anti-viral therapy (SVR). The blood samples were collected between six months and one year after achieving SVR. Using these samples, binding to rE2 and HCV-neutralization were again tested. As expected, a significant drop was observed both in binding and in neutralizing HCV following treatment (FIG. 3C-3D). Collectively, these results suggest that although the anti-HCV antibodies in resolved infections are at low levels, they are potent neutralizers. The samples that displayed high neutralization efficiency were selected for further analysis.

### Example 2: Differentiating Features Between SC and CI Antibody Repertoires

Antibody repertoires were sequenced from 28 individuals; among these are 10 HCV CI, 11 SC that displayed the highest neutralization efficiency as described above (FIG. 3B), and 7 healthy control samples. 10⁴-10⁵ unique full-length heavy chain sequences were identified for each sample (FIG. 4A).

To identify features in B-cell repertoires that are unique to CI or SC HCV infections, the usage frequency of each V and J gene segment were evaluated, as well as the CDR3 length, and the mutation frequencies across the V genes. Sequences were grouped by their V gene, J gene, and CDR3 length, clustered by genetic distance, and the frequencies within and between the clinical groups were compared. No significant differences were observed in CDR3 length, V, and J gene distributions between the clinical groups (FIG. 4B-4C-4D). V-J gene combinations, as well as V-J-CDR3 length also did not yield significant results. A similar analysis for β chains of TCRs from the same individual groups was also performed (FIG. 5A), no differences in CDR3 length, V, and J gene usage between SC and CI clinical groups were observed (FIG. 5B-5C-5D).

Next, the possibility that clusters of similar antibody sequences are enriched in either SC or CI groups was explored. To this end, the antibody sequences were grouped by V-J-CDR3 similarity. 337 clusters were identified that are different between the clinical groups by more than four samples. Of these, 165 clusters were enriched in SC samples and 172 clusters were enriched in CI samples. To narrow down the list of candidate clusters for classification, the threshold for calling a cluster enriched was increased, from four samples to five. Using this higher threshold, 13 enriched clusters were identified. Of these, 11 clusters were unique to SC, and one was unique to CI (FIG. 4E, and FIG. 6)

To evaluate the mutation frequencies between the clinical groups, the sequences were first subdivided into IgM, IgD, IgG, or IgA isotypes. No significant differences in the frequencies of the different isotypes were observed between the clinical groups (FIG. 7A). FIG. 7B displays a violin plot comparing the distribution of somatic mutation frequencies across IgA, IgD, IgG, and IgM. As expected, higher mutation numbers were observed in the IgG and IgA isotypes, compared with the IgM and IgD isotypes. No significant differences were observed in mutation numbers within each isotype between the clinical groups (FIG. 7C). Mutation numbers were also compared for each isotype across V genes between the clinical groups. Interestingly, 14 isotype-specific V genes were significantly different when comparing the clinical groups (FIG. 7D). Of these, four displayed higher mutation numbers in SC than in CI, including IGHV3-53, IGHV2-70, IGHV1-8, and IGHV3-33. The remaining ten V genes displayed lower mutation numbers in SC than in CI.

### Example 3: A Machine Learning Model Predicts Clinical Outcomes Based on the Antibody Repertoire

To determine whether a combination of features, rather than one at a time, would provide better insight into the antibody sequences that participate in the response to HCV, a machine learning approach was used, which predicts the clinical group based on a combination of features. This approach can be utilized not only as a prediction model; it can also be used as a tool to identify significant features that did not arise in the single-feature analysis.

For feature selection, frequency per sample was calculated for each cluster of sequences. To avoid false clusters that may occur due to grouping of several erroneous sequences with correct ones, rare clusters that appeared at low frequencies or in fewer than four samples were removed. Then, two samples were left out as a test set, and the model was trained on the remaining samples.

A random forest model was applied to extract the best 18 clusters (equal to the size of the training set), followed by logistic regression on the selected clusters to generate the prediction model. Finally, the model was applied to the remaining two samples and their accuracy was calculated. The process of sampling and training was repeated 100 times, to ensure that the model was not biased towards specific samples.

The final predication results, summarized in FIG. 8, indicate 91% accuracy of the prediction. As a control, when the clinical groups were randomly shuffled and the model trained, the prediction rates were 49% and 35% for the SC and CI groups, respectively (FIG. 8A, and FIG. 8B for T cells), suggesting that the high accuracy predictions were not achieved due to over fitting or another random bias of any specific sample. Therefore, sequence clusters were identified that can accurately stratify between the SC and CI samples (termed “stratifying clusters”). Of the 10 best clusters (FIG. 8C, and FIG. 9), four (IGHV3-15*IGHJ4*8**130, IGHV4-34*IGHJ6*14**103, IGHV3-23*IGHJ4*10**707, and IGHV3-23*IGHJ6*20**367) were also previously found in the single-feature comparisons (FIG. 4E).

Possible inaccuracies in multiplexed sample sequencing as a result of rare barcode impurities might cause biases. To overcome this difficulty a strict cutoff was determined. Only clones in which at most 90% of the sequences originated from one sample were used. If no cutoff had been used, the prediction precision would improve by only 2%. Lowering the cutoff to 80% decreases the precision by 13.5%. Still, a high performance of the algorithm.

Training the model for T-cell repertoires was very similar to the one for the B-cell repertoires, except that the data were categorized by identical amino acid CDR3 sequences. The average accuracy was ˜79% and 85% for the SC and CI groups, compared with 50% using shuffled labels (FIG. 8B). Of the 10 best CDR3 sequences, two sequences, CASSTAGQGLTEAFF as denoted by SEQ ID NO: 183 and CASSLGTPNEQFF (see FIG. 8D) as denoted by SEQ ID NO: 184, were also found in the single feature comparisons.

### Example 4: Differentiating the Features of HCV-Specific B-Cell Repertoires

The polyclonal nature of the immune response may impose significant background noise that interferes with characterizing the HCV-specific immune response. Thus, in order to isolate HCV-specific B cells and characterize their properties, a novel platform for the in vitro propagation and isolation of HCV-specific memory B cells was established (described in the Materials and methods). The HCV E2⁺-specific populations were separated from six CI and three SC individuals and healthy individuals as controls (FIG. 10A). The fold enrichment of HCV-specific B cells from each sample was calculated compared to the number of B cells isolated from healthy individuals, as demonstrated in FIG. 11. The fold enrichment of cells isolated from HCV-specific B cells ranged from 2 to 466 (FIG. 10A). To validate the enrichment of HCV-specific B cells, the growth media of the cells were used for the HCV-neutralization assay, which displayed higher neutralization in the CI and SC samples compared with healthy controls. Neutralization was further enhanced following separation of HCV-specific B cells (FIG. 10B).

The variable regions of the antibody's heavy chains of the HCV-specific B cells were sequenced. First, the genomic distance of the VDJ region sequences between the different samples was evaluated by the Levenshtein distance. Interestingly, some of the most closely related sequences originated from different samples. This observation implies that similar antibodies evolve in a convergent manner in different patients to bind HCV. To compare the repertoire of HCV-specific binding sequences with the total repertoire of a given donor, defined here as the “general repertoire”, sequences in the general repertoire that are similar to the specific binders were searched for Similarity was defined as having the same V gene, J gene, and CDR3 sequence that are at least 75% identical at the amino acid level. In total, 5447 clusters were detected in the general repertoire that were similar to the HCV-specific repertoire. In the specific repertoire 17 clusters were identified that were enriched in SC samples in the general repertoire, and 15 clusters that were enriched in CI samples in the general repertoire. An enriched cluster was defined as being represented in more than three samples in the cohort, and in addition, the fraction of samples in the cohort representing this cluster out of the total number of samples representing it is larger than ⅔. The lists of these clusters are presented in Table 3 and 4. A comparison between these two lists reveals that except for the V-J combination IGHV3-33*IGHJ4, which is abundant in both lists, different HCV-binding clusters are enriched in the two clinical groups.

TABLE 3
Clones detected in HCV-specific B
cell repertoire and enriched in CI

| Clone | C | CI | SC |
|---|---|---|---|
| IGHV1-18*IGHJ6*23**270 | 0 | 3 | 0 |
| IGHV3-21*IGHJ4*14**236 | 1 | 3 | 0 |
| IGHV3-21*IGHJ4*14**333 | 0 | 3 | 0 |
| IGHV3-21*IGHJ6*17**240 | 1 | 3 | 1 |
| IGHV3-30*IGHJ4*15**571 | 1 | 3 | 0 |
| IGHV3-33*IGHJ4*11**135 | 0 | 4 | 0 |
| IGHV3-33*IGHJ4*13**237 | 1 | 3 | 0 |
| IGHV3-33*IGHJ4*14**196 | 0 | 4 | 1 |
| IGHV3-33*IGHJ4*14**592 | 0 | 3 | 0 |
| IGHV3-48*IGHJ4*12**885 | 0 | 3 | 1 |
| IGHV3-48*IGHJ4*14**181 | 2 | 4 | 1 |
| IGHV3-7*IGHJ4*12**275 | 0 | 3 | 0 |
| IGHV3-7*IGHJ6*17**30 | 0 | 3 | 0 |
| IGHV4-34*IGHJ6*15**3 | 0 | 3 | 1 |
| IGHV4-34*IGHJ6*16**149 | 0 | 3 | 1 |

TABLE 4
Clones detected in HCV-specific B cell
repertoire and enriched in SC samples

| Clone | C | CI | SC |
|---|---|---|---|
| IGHV1-18*IGHJ6*15**79 | 0 | 0 | 4 |
| IGHV1-18*IGHJ6*24**16 | 0 | 0 | 3 |
| IGHV1-2*IGHJ4*13**635 | 0 | 0 | 3 |
| IGHV1-8*IGHJ6*14**18 | 0 | 0 | 3 |
| IGHV3-23*IGHJ4*14**2188 | 0 | 0 | 4 |
| IGHV3-23*IGHJ4*15**138 | 2 | 1 | 4 |
| IGHV3-23*IGHJ4*15**1489 | 0 | 0 | 3 |
| IGHV3-33*IGHJ4*12**208 | 1 | 1 | 3 |
| IGHV3-33*IGHJ4*14**185 | 0 | 0 | 3 |
| IGHV3-33*IGHJ4*15**163 | 0 | 1 | 3 |
| IGHV3-33*IGHJ4*15**208 | 0 | 2 | 3 |
| IGHV3-33*IGHJ6*17**24 | 0 | 0 | 3 |
| IGHV3-48*IGHJ4*11**104 | 0 | 0 | 4 |
| IGHV3-9*IGHJ6*18**55 | 0 | 1 | 3 |
| IGHV4-39*IGHJ5*15**201 | 0 | 2 | 3 |
| IGHV4-59*IGHJ4*11**184 | 2 | 0 | 3 |
| IGHV6-1*IGHJ6*17**20 | 0 | 0 | 5 |

Another feature that was analysed in the general repertoire, compared to the specific repertoire, is mutability. Against each specific sequence, one non-specific sequence was randomly sampled from the general repertoire. The sampled sequence contained the same V and J gene as the corresponding specific sequence. Then, sequences were grouped by isotype, and mutation numbers in the V gene were compared. Both for IgA and IgG, significantly higher mutation numbers were detected in specific compared with non-specific repertoires. For IgM, however, an opposite trend was observed (Mann Whitney test, IGA p=3.488873e-07, IGG p=6.8495Ile-08, IGM p=3.764229e-04) (FIG. 10C). This might result from the long infection period of the chronic HCV patients.

The mutation number in the HCV-specific repertoire in SC compared with CI was then evaluated. All specific sequences of SC samples were unified into one bulk, and CI samples were unified in a second bulk. Then, the sequences were grouped by isotype and the mutation numbers in the V genes were compared. The number of mutations in the SC-specific repertoire bulk was lower than that in the CI-specific repertoire (FIG. 10D). This is expected, as in CI the B cells have been through longer and repeated rounds of somatic hypermutation process which is consistent with a chronic situation that allowed the accumulation of mutations, compared with the short period of infection in SC.

The heavy chain CDR3 is the most diverse region in the antibody sequences. Therefore, conservation of amino acids in this region can highlight positions that are important for antigen binding. Therefore, conserved amino acids in the CDR3 region in the HCV-specific repertoire were searched for, compared to the general repertoire. Against each binder sequence, a random sequence with identical V, J, and CDR3 lengths were selected from the general repertoires, defined as non-binder. Then, amino acids that were conserved in binder sequences but not in non-binders were selected. Four combinations of V, J, and CDR3 lengths containing differentially conserved amino acids in CDR3 were identified (FIG. 10E). Interestingly, IGHV4-39-IGHJ6-17 contained a stretch of seven conserved residues in CDR3 and was observed in three different samples (CI56H, CI57H, and CI59H). These results imply that clones evolved independently in different subjects and converged to similar CDR3 amino acid patterns.

### Example 5: Identifying Binder Antibody Sequences Associated with HCV Infection Clearance

Next, antibodies were constructed that are associated with infection clearance. One limitation of constructing mAbs directly from bulk repertoire analysis is the pairing of heavy and light chains. The matching of heavy with light chains was performed by constructing a phage display antibody library. These antibodies contain the variable regions of both heavy and light chains as a single chain (scFv), and thus enable the design of full antibodies (Kuhn, P., et al. (2016) Proteomics. Clinical applications 10, 922-948).

In order to obtain neutralizing antibodies associated with HCV clearance, a phage display antibody library was constructed from a source of pooled PBMCs obtained from 10 SC individuals (Table 1) with a total size of 6×10⁷ individual scFvs. Another library was constructed from a source of pooled PBMCs obtained from 22 HCV CI patients, with a total size of 2×10⁷ individual scFvs. The scFv libraries were constructed by amplification of the VH and VL genes separately, and then their combinatorial assembly and cloning into a phagemid vector, as described in Materials and Methods. Next, a screening for HCV E2 binders was performed by five rounds of affinity selection with rE1E2, thereby isolating a pool of HCV-binders (2×10⁵ from the SC library and 4×10⁵ from the CI library). These libraries were screened for HCV binding antibodies by repetitive rounds of panning Six different phages that displayed 2-15-fold binding to rE2 compared with BSA as background were identified and validated (FIG. 12A).

7 antibodies were isolated from SC library and 3 antibodies from CI library. Sequence alignment of these antibodies is shown in FIG. 13 These antibodies specifically bind (FIGS. 14A and 14B) and neutralize (FIG. 14C) HCV.

Clusters of sequences were then identified from the general repertoire that were similar to the isolated scFv sequences, and the closest sequence to each scFv was selected (FIG. 12B).

In addition, Ab genes were isolated and sequenced from a panel of HCV-specific single B cells. The Ab sequences obtained from phage display, HCV-specific single B cells and repertoires of HCV-specific B cells that were sequenced as described above were clustered. Interestingly, integration of all the data revealed that two Ab sequences identified from phage libraries showed high sequence similarity to sequences of HCV-specific B cells and were also enriched in total B-cell repertoires: SC11 and SC28 in the SC repertoire. Ab SC11 demonstrated highest sequence similarity to sequence from HCV-specific B-cells and to one of the stratifying clones (98% total identity and 100% junctions identity), and most significant higher frequency in SC repertoire. Abs SC11 and SC28 were selected for construction of a full Ab and characterization.

The scFv SC11 and SC28 were then selected for constructing full-length antibodies, since they showed the highest binding to HCV E2 protein (FIG. 12A) and were the most similar to the SC general repertoires (FIG. 12B, FIG. 15). The closest cluster to scFv SC28 was IGHV4-39*IGHJ4*13*861, which was detected in the repertoires of four out of nine SC samples, and the closest cluster to scFv SC11 was IGHV6-1*IGHJ6*17**20, which was detected in repertoires of five out of nine SC samples (FIG. 4E). Both clusters were not detected in CI repertoires. Cluster IGHV6-1*IGHJ6*17**20 was also enriched in the HCV-specific repertoire (Table 4). Lineage trees revealed that the closest sequences to SC11 and SC28 are positioned relatively high in the tree (FIG. 12C-12D), suggesting that these sequences appeared earlier during the infection.

### Example 6: Construction of Broadly Neutralizing Antibodies Associated with HCV Infection Clearance

Full-length antibodies were constructed from scFvs SC11 and SC28.

Additional full-length antibodies were constructed with identical light chains, but with heavy chains of one of the nearest sequences to the heavy chains of scFv SC11 and scFv SC28 from the general repertoires (RMS11 (VH16618) and RMS28 (VH510520), respectively). The binding specificities of these four antibodies to HCV rE2 protein were evaluated. More than 35-fold higher binding signals in antibodies RMS11 and RMS28 were observed than with antibodies SC11 and SC28 (FIG. 16A). To further characterize the binding capacity of RMS11 and RMS28, the binding of these antibodies were compared to a well-characterized panel of mAbs, including CBH-4B, CBH-4D, HC-1, HC-11, CBH-7, HC84.22, HC84.26, HC33.1, and HC33.4, which are representative E2 antigenic domain A-E antibodies (Pierce, B. G., et al. (2016) Proceedings of the National Academy of Sciences of the United States of America) and were reviewed previously (Kong, L., et al. (2015) Current opinion in virology 11, 148-157; Ball, J. K., et al. (2014) Antiviral research 105, 100-111). Protein indicated binding capacity of RMS11 and RMS28 was comparable to the well-defined panel (FIG. 16B). To evaluate neutralization breadth, neutralization assays were performed with these antibodies across all HCV genotypes using a panel of infectious HCVcc containing envelope proteins from HCV genotypes 1-7 (Gottwein, J. M., et al. (2009) Hepatology Baltimore, Md. 49, 364-377). The percent neutralization was calculated as the percent reduction in FFU compared with virus incubated with an irrelevant control antibody RO4 (Hadlock, K. G., et al. (2000) Journal of virology 74, 10407-10416). Antibodies RMS11 and RMS28 efficiently neutralized all seven HCV genotypes, including genotype three which was less efficiently neutralized by previous panels of HCV antibodies including a recent SC panel [8] pointing out their exceptionally high neutralization breadth (FIG. 16B-16C).

1. An isolated monoclonal antibody or any antigen-binding fragment thereof which binds to hepatitis C virus E2 protein (HCV E2), wherein said antibody is selected from a group consisting of:
a. a monoclonal antibody comprising a heavy chain complementarity determining region (CDRH) 1 denoted by SEQ ID NO. 158, CDRH2 denoted by SEQ ID NO. 159, CDRH3 denoted by SEQ ID NO. 160, and the light chain complementarity determining region (CDRL) 1 denoted by SEQ ID NO. 163, a CDRL2 denoted by SEQ ID NO. 65, and a CDRL3 denoted by SEQ ID NO. 165, or a variant thereof;
b. a monoclonal antibody comprising a CDRH1 denoted by SEQ ID NO. 168, CDRH2 denoted by SEQ ID NO. 169, CDRH3 denoted by SEQ ID NO. 170, and a CDRL1 denoted by SEQ ID NO. 173, a CDRL2 denoted by SEQ ID NO. 65, and a CDRL3 denoted by SEQ ID NO. 175, or a variant thereof;
c. a monoclonal antibody comprising a CDRH1 denoted by SEQ ID NO. 108, CDRH2 denoted by SEQ ID NO. 109, CDRH3 denoted by SEQ ID NO. 110, and a CDRL1 denoted by SEQ ID NO. 113, a CDRL2 denoted by SEQ ID NO. 65, and a CDRL3 denoted by SEQ ID NO. 115, or a variant thereof;
d. a monoclonal antibody comprising a CDRH1 denoted by SEQ ID NO. 78, CDRH2 denoted by SEQ ID NO. 79, CDRH3 denoted by SEQ ID NO. 80, and a CDRL1 denoted by SEQ ID NO. 83, a CDRL2 denoted by SEQ ID NO. 65, and a CDRL3 denoted by SEQ ID NO. 85, or a variant thereof;
e. a monoclonal antibody comprising a CDRH1 denoted by SEQ ID NO. 59, CDRH2 denoted by SEQ ID NO. 60, CDRH3 denoted by SEQ ID NO. 61, and a CDRL1 denoted by SEQ ID NO. 64, a CDRL2 denoted by SEQ ID NO. 65, and a CDRL3 denoted by SEQ ID NO. 66, or a variant thereof;
f. a monoclonal antibody comprising a CDRH1 denoted by SEQ ID NO. 69, CDRH2 denoted by SEQ ID NO. 70, CDRH3 denoted by SEQ ID NO. 182, and a CDRL1 denoted by SEQ ID NO. 73, a CDRL2 denoted by SEQ ID NO. 74, and a CDRL3 denoted by SEQ ID NO. 75, or a variant thereof;
g. a monoclonal antibody comprising a CDRH1 denoted by SEQ ID NO. 88, CDRH2 denoted by SEQ ID NO. 89, CDRH3 denoted by SEQ ID NO. 90, and a CDRL1 denoted by SEQ ID NO. 93, a CDRL2 denoted by SEQ ID NO. 65, and a CDRL3 denoted by SEQ ID NO. 95, or a variant thereof;
h. a monoclonal antibody comprising a CDRH1 denoted by SEQ ID NO. 98, CDRH2 denoted by SEQ ID NO. 99, CDRH3 denoted by SEQ ID NO. 100, and a CDRL1 denoted by SEQ ID NO. 103, a CDRL2 denoted by SEQ ID NO. 104, and a CDRL3 denoted by SEQ ID NO. 105, or a variant thereof;
i. a monoclonal antibody comprising a CDRH1 denoted by SEQ ID NO. 118, CDRH2 denoted by SEQ ID NO. 119, CDRH3 denoted by SEQ ID NO. 120, and a CDRL1 denoted by SEQ ID NO. 123, a CDRL2 denoted by SEQ ID NO. 124, and a CDRL3 denoted by SEQ ID NO. 125, or a variant thereof;
j. a monoclonal antibody comprising a CDRH1 denoted by SEQ ID NO. 128, CDRH2 denoted by SEQ ID NO. 129, CDRH3 denoted by SEQ ID NO. 130, and a CDRL1 denoted by SEQ ID NO. 133, a CDRL2 denoted by SEQ ID NO. 134, and a CDRL3 denoted by SEQ ID NO. 135, or a variant thereof;
k. a monoclonal antibody comprising a CDRH1 denoted by SEQ ID NO. 138, CDRH2 denoted by SEQ ID NO. 139, CDRH3 denoted by SEQ ID NO. 140, and a CDRL1 denoted by SEQ ID NO. 143, a CDRL2 denoted by SEQ ID NO. 65, and a CDRL3 denoted by SEQ ID NO. 145, or a variant thereof; and
l. a monoclonal antibody comprising a CDRH1 denoted by SEQ ID NO. 148, CDRH2 denoted by SEQ ID NO. 149, CDRH3 denoted by SEQ ID NO. 150, and a CDRL1 denoted by SEQ ID NO. 153, a CDRL2 denoted by SEQ ID NO. 104, and a CDRL3 denoted by SEQ ID NO. 155, or a variant thereof.

2. An isolated monoclonal antibody or any antigen-binding fragment thereof which binds to HCV E2, wherein said antibody comprises a heavy chain variable region and a light chain variable region, wherein said heavy chain variable region is encoded by a nucleic acid sequence which is at least 70% identical to the nucleic acid sequence denoted by SEQ ID NO. 156, SEQ ID NO. 166, SEQ ID NO. 106, SEQ ID NO. 76, SEQ ID NO. 57, SEQ ID NO. 67, SEQ ID NO. 86, SEQ ID NO. 96, SEQ ID NO. 116, SEQ ID NO. 126, SEQ ID NO. 136, SEQ ID NO. 146, and wherein said light chain variable region is encoded by a nucleic acid sequence which is at least 70% identical to SEQ ID NO. 161, SEQ ID NO. 171, SEQ ID NO. 111, SEQ ID NO. 81, SEQ ID NO. 62, SEQ ID NO. 71, SEQ ID NO. 91, SEQ ID NO. 101, SEQ ID NO. 121, SEQ ID NO. 131, SEQ ID NO. 141, SEQ ID NO. 151.

3. The isolated monoclonal antibody according to claim 1 or 2, wherein said antibody comprises a heavy chain variable region comprising the amino acid sequence denoted by SEQ ID NO. 157, SEQ ID NO. 167, SEQ ID NO. 107, SEQ ID NO. 77, SEQ ID NO. 58, SEQ ID NO. 68, SEQ ID NO. 87, SEQ ID NO. 97, SEQ ID NO. 117, SEQ ID NO. 127, SEQ ID NO. 137, SEQ ID NO. 147, or a variant thereof and a light chain variable region comprising the amino acid sequence denoted by SEQ ID NO. 162, SEQ ID NO. 172, SEQ ID NO. 112, SEQ ID NO. 82, SEQ ID NO. 63, SEQ ID NO. 72, SEQ ID NO. 92, SEQ ID NO. 102, SEQ ID NO. 122, SEQ ID NO. 132, SEQ ID NO. 142, SEQ ID NO. 152, or a variant thereof.

4. The isolated monoclonal antibody according to claim 1 or 2, wherein said antibody is selected from a group consisting of:
a. a monoclonal antibody comprising a heavy chain variable region comprising the amino acid sequence denoted by SEQ ID NO. 157 or a variant thereof and a light chain variable region comprising the amino acid sequence denoted by SEQ ID NO. 162 or a variant thereof; and
b. a monoclonal antibody comprising a heavy chain variable region comprising the amino acid sequence denoted by SEQ ID NO. 167 or a variant thereof and a light chain variable region comprising the amino acid sequence denoted by SEQ ID NO. 172 or a variant thereof.

5. The isolated monoclonal antibody according to claim 1, wherein said antibody is a human antibody.

6. (canceled)

7. An isolated nucleic acid molecule comprising a nucleotide sequence encoding an antibody or any antigen-binding fragment thereof according to claim 1.

8. An expression vector or a host cell comprising the isolated nucleic acid molecule according to claim 7.

9.-10. (canceled)

11. A pharmaceutical composition comprising as an active ingredient the isolated monoclonal antibody or any antigen-binding fragment thereof according to claim 1 and a pharmaceutically acceptable carrier, excipient or diluent.

12. The pharmaceutical composition according to claim 11, wherein said composition further comprises an additional anti-HCV agent.

13. A method of prophylaxis, treatment or amelioration of HCV infection comprising administering to a subject in need thereof a therapeutically effective amount of the isolated monoclonal antibody or any antigen-binding fragment thereof according to claim 1.

14. The method according to claim 13, wherein said method further comprises administering to a subject in need thereof an additional anti-HCV agent.

15. The method according to claim 14, wherein said antibody is administered at a therapeutically effective amount of 10-1000 μg/kg.

16. A method of detecting HCV in a biological sample obtained from a subject, said method comprising:
(a) contacting said biological sample with the isolated monoclonal antibody or any antigen-binding fragment thereof according to claim 1, wherein said monoclonal antibody is labeled with a detectable marker; and
(b) detecting said isolated monoclonal antibody or any antigen-binding fragment thereof;
wherein the presence of said isolated monoclonal antibody or any antigen-binding fragment thereof indicates the presence of HCV in said biological sample.

17. A kit for detecting HCV comprising:
(a) at least one labeled isolated monoclonal antibody or any antigen-binding fragment thereof according to claim 1;
(b) means for detection of said labeled isolated monoclonal antibody; and optionally
(c) instructions for use of said kit.

18. A method of preparing neutralizing anti HCV scFv antibodies associated with HCV clearance, comprising the steps of:
a. constructing a phage display scFv antibody library from peripheral blood mononuclear cells (PBMC) obtained from Spontaneous clearer (SC) individuals;
b. identifying at least one HCV E2 binder;
c. comparing the sequence of the at least one HCV E2 binder with the sequences in stratifying clusters from a general repertoire that are associated with HCV clearance;
d. selecting an scFv sequence with high similarity to a sequence in a stratifying cluster from said general repertoire that is associated with HCV clearance.

19. The method of claim 18 wherein the scFv library construction comprises the steps of:
a. amplification of the Heavy chain variable region (V_{H}) and Light chain variable region (V_{L}) genes separately;
b. combinatorial assembly of the V_{H} and V_{L} genes; and
c. cloning into a phagemid vector.

20. The method of claim 18 wherein the identification of the at least one HCV E2 binder comprises screening for phages that bind to rE2.

21. The method of claim 18 wherein the stratifying clusters are selected from the clusters denoted in Tables 3 and 4.

22. The method of claim 18 further comprising the step of converting the scFv to full-length antibodies.

23. The method of claim 22 wherein the full-length antibodies comprise a light chain of the selected scFv antibody and a heavy chain of one of the sequences that show high similarity to the scFv heavy chain from the general repertoire.

24. (canceled)
